(19) United States
(12) Patent Application Publication
(10) Pub. No.: US 2018/0168729 A1
(43) Pub. Date: Jun. 21, 2018

(54) CATHETER SYSTEM AND METHOD OF ABLATING A TISSUE

(71) Applicant: Lazcath Pty Ltd, Mosman Park (AU)

(72) Inventors: Peter Pratten, Mosman Park (AU); Rukshen Weerasooriya, Mosman Park (AU); Kamal Alameh, Mosman Park (AU)

(73) Assignee: Lazcath Pty Ltd, Mosman Park (AU)

(21) Appl. No.: 15/577,162

(22) PCT Filed: May 25, 2016
(86) PCT No.: PCT/AU2016/050403

§ 371 Date: Nov. 27, 2017

(30) Foreign Application Priority Data
May 25, 2015 (AU) 2015901911

(51) Int. Cl. A61B 18/24; A61B 18/08; A61N 1/362; A61B 18/14

(52) U.S. Cl. CPC A61B 18/24; A61B 18/082; A61B 5/0066; A61B 18/1492; A61N 1/362

(57) ABSTRACT

A catheter system for ablating a tissue portion of a body and visualising the ablation in real time, the system comprising a means for generating an optical imaging beam, a catheter including a catheter tip assembly comprising an array of first optical fibres for carrying the optical imaging beam and an ablating means, wherein the catheter tip assembly is adapted to direct said beam onto the tissue portion and capture a reflected portion of the optical imaging beam from the tissue portion. The system further includes a first switching means for switching the optical imaging beam between a plurality of the first optical fibres in the array and a means for processing the reflected portion of the optical imaging beam to account for variations in length between the first optical fibres.

### FIELD OF THE INVENTION

The present invention relates to a catheter system and to a method of ablating a tissue in a subject and visualising the ablation process in real time via an image of the tissue.

### BACKGROUND TO THE INVENTION

Cardiovascular disease is a leading cause of mortality and disability in the world.

Cardiovascular disease may account for around 30% of deaths in some regions, half of which may be due to heart failure, e.g., progressive alteration of cardiac contraction, which is entirely dependent on prior electrical activation. A substantial number of cases of heart failure are secondary to or aggravated by electrical dysfunctions: e.g., uncoordinated contraction (mechanical dyssynchrony) and heart arrhythmias, the most frequent of which is atrial fibrillation (AF).

Cardiovascular disease, specifically in the form of AF, is a major cause of cardioembolic strokes, and can diminish heart function to the extent that physical fitness, and capacity to perform routine duties or work, are impacted. Atrial fibrillation (AF), in its paroxysmal form, is mainly initiated by ectopic foci from the pulmonary veins.

Atrial Flutter, similarly debilitating, is usually initiated by aberrant foci within the wall of the right atrium. Ventricular arrhythmias are sometimes also diagnosable and treatable using the techniques described below. Cardiovascular disease may be treatable with curative ablation therapy or treatment. Ablation therapy may restore electrical synchrony (resynchronization) to provide a more homogeneous contraction of the heart. Structural abnormalities that may cause cardiac death may be treatable with ablation. Treatment of AF may involve an ablative therapy that includes isolation and exclusion of venous sources. There are rapidly growing indications (0 in 1990 versus 200,000 in 2010) for interventional ablation therapy for AF.

Currently, interventionist cardiac electro-physiologists (CPEs) may be using minimally invasive thin hollow flexible catheters that are each equipped with a radio-frequency (RF) heater for tissue ablation and an electronic sensor that detects contact between blood vessel walls and the catheter tip assembly. Existing catheters may be inserted through the Inferior Vena Cava via groin access, or through Superior Vena Cava via arm or neck access fed in to the right atrium, then through the intra-atrial septum to pulmonary vein areas in the left atrium. Key advantages of using catheters over open surgery include: faster recovery of RF ablated patients reverted to sinus rhythm (e.g., in hours or days instead of months); less operative time; less morbidity and mortality; and lower cost. Current CPE techniques may also include insertion of a separate ultrasound catheter to determine the thickness of intra-body tissue, e.g., after ablation.

However, current CPE catheter techniques may impose problematic limitations on catheter-based treatment.

One source of problems is the RF radiation. RF ablation may have fundamental limitations, including the need for excellent electrode-tissue contact, which may lead to superficial lesions, and the difficulty of focusing the RF radiation into a small area because RF beams are not very spatially coherent: the RF energy typically heats in a sphere from point of application causing tissue burn injuries to surrounding blood or other tissues not intended as specific targets for ablation.

Another source of problems is the difficulty in determining tissue depth before and after ablation, e.g., to determine how much tissue needs to be removed, how has been removed by a burn, and/or how much tissue remains after a burn. Current AF procedures do not allow the ablated tissues to be accurately reviewed at during the procedure. The RF ablation is executed using empirical evidence relative to power, tissue contact, time of dwell on the tissue, and the knowledge and judgement of these by the operator. Incomplete ablation may lead to failure or to early post-operative arrhythmia, in which case it may be necessary for the patient to undergo another ablation procedure. A full thickness of tissue needs to be ablated to electrically insulate the normal heart rhythm generated from the sinoatrial node (SAN) from the over-riding aberrant signal arising from elsewhere in the heart (e.g., in AF this usually comes from points near the junction of the pulmonary veins with the left atrium, and in Atrial Flutter the aberrant rhythm usually arises in the right atrium). Incompletely ablated tissues may lead to the early post-operative arrhythmia due to lack of complete full electrical insulation of the SAN once the oedema and tissue damage has healed (e.g., in around 43-59% of patients, with more than 90% of these arrhythmias occurring up to three months after surgery).

Another source of problems is operation, coordination and handling of the three or more separate catheters required simultaneously in the heart during the procedure to provide (1) internal cardiac monitoring and pacing, (2) intracavity mapping (using a multielectrode mapping catheter), (3) ablation, and (4) if required, but not uniformly employed due to its inherent inaccuracy, an ultrasound catheter in the heart. The presence of multiple catheters in the heart at one time increases the risk of embolism and stroke due to potential formation of clots or dislodgement of tissue from the heart or vascular wall.

Another related problem is the small but significant risk of penetrating the heart wall, potentially causing escape of blood into pericardium or oesophagus, e.g., due to miscalculation of the heart-wall thickness at the point of burning.

It is desired to address or ameliorate one or more problems, disadvantages or limitations associated with the existing techniques, or to at least provide a useful alternative.

### SUMMARY OF THE INVENTION

The present invention provides a catheter system for ablating a tissue portion of a body and visualising the ablation in real time, the system comprising:
(i) a means for generating an optical imaging beam;
(ii) a catheter including a catheter tip assembly comprising:
   (a) an array of first optical fibres for carrying the optical imaging beam; and
   (b) an ablating means;
   wherein the catheter tip assembly is adapted to direct said beam onto the tissue portion and capture a reflected portion of the optical imaging beam from the tissue portion;
(iii) a first switching means for switching the optical imaging beam between a plurality of the first optical fibres in the array; and
(iv) a means for processing the reflected portion of the optical imaging beam to account for variations in length between the first optical fibres.

The present invention also provides a method of ablating a tissue in a subject and visualising the ablation process in real time via an image of the tissue, the method comprising the steps of:
(i) positioning, adjacent to the tissue, a catheter tip assembly comprising:
   (a) an array of first optical fibres for carrying the optical imaging beam; and
   (b) an ablating means;
   wherein the catheter tip assembly is adapted to direct said beam onto the tissue portion and capture a reflected portion of the optical imaging beam from the tissue portion;
(ii) actuating the ablating means and simultaneously directing said beam onto the tissue;
(iii) actuating a first switching means to switch the optical imaging beam between a plurality of the first optical fibres and capturing the optical imaging beam reflected from the tissue;
(iv) adjusting the captured optical imaging beam reflected from the tissue to account for variations in length between the first optical fibres; and
(v) using the adjusted captured optical imaging beam from step (iv) to create the image of the tissue.

The invention may be applied to heat ablation employing radio frequency current (RF) from the catheter tip assembly or incorporate the use of fibre-optic transmission of heat ablation using laser energy such as infrared laser energy from the catheter tip assembly.

### BRIEF DESCRIPTION OF DRAWINGS

Preferred embodiments of the present invention are hereinafter described, by way of non-limiting example only, with reference to the accompanying drawings, in which:

FIG. 1 is a schematic diagram of a catheter system for treatment of intrabody tissues;

FIG. 2A is a schematic diagram of an optical conduit or fibre and a catheter tip assembly with an end window (referred to as “an end-window tip”) of the catheter system—note that the length of the trailing end of the fibre is not to scale and extends to connect with the rest of the catheter system—this comment also applies to the corresponding trailing end of the fibre depicted in FIGS. 2B-2D and 3A-3D;

FIG. 2B is a schematic diagram of the end-window catheter tip assembly directing an imaging beam from the optical conduit to a tissue portion, and back from the tissue portion to the optical conduit, through the end window;

FIG. 2C is a schematic diagram of the end-window catheter tip assembly directing an ablating beam from the optical conduit or fibre to the tissue portion through the end window;

FIG. 2D is a schematic diagram of a temperature and/or pressure sensing component in the end-window catheter tip assembly reflecting a sensing beam from the optical conduit or fibre back along the optical conduit or fibre;

FIG. 3A is a schematic diagram of the optical conduit or fibre and the catheter tip assembly with a side window (referred to as “a side-window tip”) of the catheter system;

FIG. 3B is a schematic diagram of the side-window catheter tip assembly directing the imaging beam from the optical conduit or fibre to the tissue portion, and back from the tissue portion to the optical conduit or fibre, through the side window;

FIG. 3C is a schematic diagram of the side-window catheter tip assembly directing the ablating beam from the optical conduit or fibre to the tissue portion through the side window;

FIG. 3D is a schematic diagram of the sensing component in the side-window catheter tip assembly reflecting the sensing beam from the optical conduit or fibre back along the optical conduit or fibre;

FIG. 4 is a schematic diagram of the catheter system including a plurality of optical fibres and an optical switch;

FIG. 5A is a schematic diagram of the imaging beam forming a plurality of spots;

FIG. 5B is a schematic diagram of the ablating beam forming a plurality of spots;

FIG. 6 is a schematic diagram of a fibre-optic (FO) catheter with an internal rotary joint between the optical conduit and the catheter tip assembly;

FIGS. 7A and 7B are illustrations of the catheter system ablating a tissue portion;

FIG. 8A is a schematic illustration (cross section through A-A in FIG. 8B) of a catheter tip assembly for a catheter system according to one embodiment of the present invention;

FIG. 8B is an end view of the catheter tip assembly in FIG. 8A;

FIG. 9 is a schematic illustration of a catheter system according to another embodiment of the present invention demonstrating the ablating laser beam;

FIG. 10 is a schematic layout of a catheter system according to another embodiment of the present invention;

FIG. 11 is a schematic layout of a catheter system according to another embodiment of the present invention;

FIG. 12A is an end view of a catheter tip assembly for a catheter system, adapted for laser ablation, according to an embodiment of the present invention;

FIG. 12B is a schematic side view showing the optical ablating beam being projected onto the surface of a tissue from the catheter tip assembly in FIG. 12A;

FIG. 12C is a schematic side view showing the optical imaging beam being projected onto the surface of a tissue from the catheter tip assembly in FIG. 12A;

FIG. 13A is a schematic side view in cross section of a catheter tip assembly for a catheter system, adapted for RF ablation, showing the optical imaging beam being projected from two of six optical fibres in the catheter tip assembly through the catheter jacket;

FIG. 13B is a schematic end view of the catheter tip assembly in FIG. 13A, without the catheter jacket, aligned with a cross sectional view through A-A; and

FIG. 14 is a schematic perspective view showing the interior of another catheter tip assembly for a catheter system, adapted for RF ablation.

### DETAILED DESCRIPTION OF THE INVENTION

The present invention provides a catheter system for ablating a tissue portion of a body and visualising the ablation in real time, the system comprising:
(i) a means for generating an optical imaging beam;
(ii) a catheter including a catheter tip assembly comprising:
   (a) an array of first optical fibres for carrying the optical imaging beam; and
   (b) an ablating means;
   wherein the catheter tip assembly is adapted to direct said beam onto the tissue portion and capture a reflected portion of the optical imaging beam from the tissue portion;
(iii) a first switching means for switching the optical imaging beam between a plurality of the first optical fibres; and
(iv) a means for processing the reflected portion of the optical imaging beam to account for variations in length between the first optical fibres.

Preferably, the means for generating an optical imaging beam is an optical coherence tomography (OCT) system. In this regard, the optical imaging beam may be a tomography beam capable of generating tomographic data. Alternatively, the optical imaging beam may be a diagnostic beam when it is used to generate diagnostic data of a 2D or 3D region of the tissue portion.

When the means for generating an optical imaging beam is an OCT system, the system may be configured to operate based on a frequency domain approach. Even more preferably, the system operates as a swept source OCT (SS-OCT). SS-OCT is adapted to perform a rapid, continuous sweep of the target tissue using a broad, longer wavelength optical imaging beam and can give improved visualisation of the target tissue including a greater depth of visualisation into the tissue e.g. 5-6 mm.

Preferably, the means for generating an optical imaging beam is able to generate an optical imaging beam at a selected wavelength from 700-3000 nm, 1000-2500 nm or 1750-2250 nm e.g. about 930 nm or about 2000 nm.

The array of first optical fibres may comprise at least 2-6, 2-10 or 2-20 optical fibres. In one form of the invention the array of first optical fibres comprises 6 optical fibres.

By employing an array of fibres in the catheter tip assembly to carry the optical imaging beam and/or capture the reflected portion thereof and using the fibres appropriately the catheter system of the subject invention is able to visualise the ablation without needing the catheter tip assembly to be positioned precisely relative to the tissue portion being ablated. In this regard, provided a subset of the fibres in the array are positioned to receive a reflected portion of the optical imaging beam image the ablation can be visualised. Furthermore, the system is able to process data generated from individual fibres in the array in various ways to optimise visualisation of the ablation. For example, the system may only use data from a subset of the fibres to visualise the ablation.

The array of first optical fibres may be located inside, outside or around the ablating means. Preferably, the array of first optical fibres is arranged in a circular formation.

Preferably, at least one of the first optical fibres further comprises an optical directing component. Even more preferably, half of the first optical fibres further comprise an optical directing component.

The optical directing component may be a separate component in optical communication with the first optical fibre or provided integrally with the first optical fibre.

The catheter tip assembly may further comprise a platform member located in the catheter tip assembly and the first optical fibres may terminate at apertures formed in the platform member that comprise the optical directing component.

The optical directing component may be adapted to deflect a beam emanating from the first optical fibre by less than or equal to 90°, about 30°-60° or about 45°.

Preferably, the optical directing component is a lens such as a prism. When the optical directing component is a prism it may be cylindrical. In one form of the invention the lens is a GRIN lens.

The optical directing component may be provided as a separate component in optical communication with the said fibres. Alternatively, the optical directing component may be provided integrally with the said fibres. For example, when the said beams are carried in an optical conduit such as an optical fibre, the optical directing component may be provided integrally with the fibre. Alternatively, the catheter tip assembly may comprise a platform member located in the catheter tip assembly and the optical conduits terminate at apertures formed in the platform member that comprise the optical directing component.

When the ablating means is an optical ablating beam, the optical directing component for the ablating beam may be adapted to cause divergence or collimation of the ablating beam. The amount of divergence or collimation of the ablating beam may be selected to adjust the size of the area to be ablated.

Preferably, the optical directing component for the optical imaging beam is adapted to cause convergence or focussing of the beam.

Preferably, the optical directing component is multi-directional.

Preferably, the ablating means is located centrally relative to the array of first optical fibres.

Preferably, the ablating means is an optical ablating means such as a second optical fibre or array of second optical fibres adapted to carry an ablating beam. When the ablating means comprises an array of second optical fibres it may comprise at least 2-4 optical fibres.

By employing an array of fibres in the catheter tip assembly for the optical ablating means and using the fibres appropriately the catheter system of the subject invention is able to perform an ablation without needing the catheter tip assembly to be positioned precisely relative to the tissue portion being ablated. In this regard, provided a subset of the fibres for performing the ablation are well positioned the ablation can be performed. For example, the system may perform the ablation using a subset of the available fibres. Preferably, the optical ablating means is adapted to carry and or generate an optical ablating beam with a wavelength of about 808-980 nm, 800-1000 nm or 1064 nm.

The means for generating an optical ablating beam can be an ablating system such as a fibre laser system that is able to generate an ablating laser beam at a selected wavelength for ablating tissue.

Preferably, the optical ablating means further comprises an optical directing component. When the optical ablating means comprises an array of second optical fibres it is preferred that 50-75% of the second optical fibres further comprise an optical directing component.

The optical directing component may be a separate component in optical communication with the optical ablating means or second optical fibre. Alternatively, the optical directing component is provided integrally with the optical ablating means or second optical fibre. When the catheter tip assembly further comprises a platform member located in the catheter tip assembly, the optical ablating means or second optical fibre may terminate at aperture(s) formed in the platform member that comprises the optical directing component.

Preferably, the optical directing component for the optical ablating means is adapted to deflect or divert a beam emanating from the second optical fibre by less than or equal to 90°, about 30°-60°, or about 45°. Preferably, the optical directing component can be controlled to adjust the amount of deflection or diversion of the beam.

The ablating means may also be a heat source such as a radio frequency ablating means. In this regard, the ablating means may comprise a member heated by electricity or radio frequency waves such as high frequency alternating current, e.g. high frequency alternating current in the range of 350-500 kHz. Preferably, the member heated by electricity or radio frequency waves is located at the leading end of the catheter tip assembly.

When the catheter tip assembly comprises an ablating means in the form of a radio frequency ablating means the catheter tip assembly may be adapted to act as a heat or radio frequency disseminator. In one particular form of the invention involving a radio frequency ablating beam, the catheter tip assembly may comprise a surface formed of a suitable material such as gold that is adapted to contact the target tissue during the ablation process.

The catheter tip assembly may further comprise a means for carrying an optical sensing beam for a sensing component in the catheter tip assembly. Preferably, the sensing component comprises a pressure sensor and/or a temperature sensor. In one form of the invention said means comprises an array of optical fibres. Preferably, the optical sensing beam has a wavelength of 1300-1550 nm.

The arrays described herein may be arranged in a variety of cross sectional patterns. Preferably, the array of first optical fibres are located towards the outside of the array of second optical fibres. Even more preferably, the array of second optical fibres and hence the ablating means is located between at least two first optical fibres. In one particular form of the invention, a plurality of first optical fibres surround the second optical fibre in a generally circular arrangement and the second optical fibre is located at the centre of the circular arrangement.

The optical fibres may be supported from or terminate at a platform member or chip member located in the catheter tip assembly. Preferably, the platform member or chip member comprises a plurality of apertures, each aperture being for a different optical fibre.

Preferably, the reflected portion of the optical imaging beam is captured in the at least one of the first or second optical fibres. In this regard, it is preferred that at least one of the first or second optical fibres is multidirectional.

The first switching means may be adapted to switch the optical imaging beam sequentially between a plurality of the first optical fibres. When the ablating means is an optical ablating means, the first switching means may be adapted to switch the optical imaging beam sequentially and/or preferentially between a plurality of the first optical fibres and the optical ablating means.

Preferably, the means for processing the reflected portion of the optical imaging beam to account for variations in length between the first optical fibres comprises a reference data source.

The reference data source may comprise a second array of first optical fibres for carrying the optical imaging beam.

Preferably, the means for processing the reflected portion of the optical imaging beam to account for variations in length between the first optical fibres in the array comprises a second switching means for switching the optical imaging beam between a plurality of the first optical fibres in the second array.

Preferably, the second array is located outside the body. In this regard, data from the second array can be used to calibrate the data feed from the first array including accounting for variations in length of optical fibres in the catheter. In particular, the data from the second array can be fed back to an electronic controller to adjust the signal from each catheter fibre in first array.

Thus, it will be appreciated that the catheter system may compensate for variations in the length of optical fibres used in the system. In this regard, when using OCT in real time, small variations between the lengths of fibres used in the components can significantly degrade the quality of the OCT generated images. Preferably, the means for compensating for variations in the length of optical fibres used in the system involves calibrating the length of each fibre used in the system.

The means for processing the reflected portion of the optical imaging beam to account for variations in length between the first optical fibres may also comprise software including an algorithm that calibrates the reflected portion of the optical imaging beam based on the reference data source.

The catheter tip assembly may comprise at least one aperture for the optical fibres described herein. In this regard, the first optical fibres and the optical fibres associated with optical ablating means may terminate at or adjacent to a respective aperture in the catheter tip assembly. Preferably, the at least one aperture comprises a glass covering.

The catheter tip assembly may further comprise a sensing component. Preferably, the sensing component comprises a pressure sensor and/or a temperature sensor.

Preferably, the catheter tip assembly comprises a body defining a side a trailing end and a leading end and the optical fibres terminate at a point located therebetween. Even more preferably the optical directing component is located at a point therebetween. Preferably, the trailing end comprises a means for receiving the said conduits or optical fibres and the side and or leading end is physically closed but permeable to optical beams. For example the side or leading end of the catheter tip assembly may comprise an aperture formed of glass or some other suitable material. Preferably, the aperture has an inside diameter or width of less than or equal to 5, 4, 3, 2.5 or 2 mm or about 0.25-0.5 mm less than the outer diameter or width of the catheter tip assembly (see below).

When the catheter tip assembly comprises an aperture in its side it may further comprise a beam steerer for directing a beam through the aperture on the side.

Preferably, the body of the catheter tip assembly comprises a circular shaped cross section. Preferably, the body of the catheter tip assembly has an outside width or diameter of less than or equal to 5, 4, 3, 2.5 or 2 mm.

The catheter tip assembly may further comprise at least one magnet. Preferably, the catheter tip assembly comprises three magnets. Preferably, the magnets are located at or adjacent the leading end of the catheter tip assembly. When present, the magnets can be used to help guide the catheter tip assembly during use. However, the catheter system of the present invention can employ other guidance systems such as guide wires and other conventional guidance systems.

As mentioned above, the ablating means may be a radio frequency ablating means or an optical ablating beam. When the ablating means is an optical ablating means, the catheter tip assembly may further comprise a means for emitting radio-frequency waves. In this regard, the catheter tip assembly may further comprise a fourth conduit for a radio frequency ablating beam, the catheter tip assembly being adapted to direct said radio frequency ablating beam onto a tissue portion of a body. Thus, it will be appreciated that the catheter tip assembly may comprise either or both of an optical ablating beam and a radio frequency ablating means.

The catheter tip assembly may further comprise a cooling system to maintain the temperature of the catheter tip assembly at a desirable level. Preferably, the cooling system comprises a water conduit. The cooling system is particularly useful when the ablating means generates heat.

The catheter tip assembly may further comprise a flushing system for removing debris from the ablation site. Preferably, the flushing system comprises a fluid channel for carrying saline or the like.

The catheter tip assembly may further comprise a means for emitting ultrasound waves. In this regard, the catheter tip assembly may further comprise a conduit for an ultrasound beam, the catheter tip assembly being adapted to direct said ultrasound beam onto a tissue portion of a body to assist with imaging the tissue prior to ablation. Preferably, the conduit for the ultrasound beam is a fibre optic conduit.

The catheter tip assembly may also comprise a leading end including indium tin oxide. In this regard, by varying the proportions of the indium, tin and oxygen in the indium tin oxide different properties may be conferred that are useful in respect of the present invention. Preferably, the indium tin oxide leading end comprises a light transparent electrode. Even more preferably the indium tin oxide leading end comprises an infra red light transparent electrode. In one particular form of the invention the indium tin oxide leading end of the catheter tip assembly has an IR transparency of at least 75%.

The catheter system may be configured to lie inside a sleeve catheter in a body during insertion and/or placement. However, it is preferred for the catheter system to be used without a sleeve catheter or with only limited use of a sleeve catheter to allow initial insertion of the catheter.

Any of the optical conduits or fibres herein may be adapted to carry a reflected portion of at least one of the said beams away from the catheter tip assembly. Furthermore, a single optical fibre may be operable to carry multiple beams. For example, the optical fibres may comprise at least one optical fibre such as a single optical fibre. Alternatively, the at least one optical fibre may comprise a plurality of optical fibres.

When the at least one optical fibre comprises a plurality of optical fibres each fibre may carry a plurality of the said beams. Alternatively, at least one fibre may carry different ones of the said beams. In another form of the invention a least one fibre may carry two of the said beams and at least one fibre may carry another of the said beams.

Preferably, the at least one optical fibre is configured to have one or more optical transmission bands selected to carry a plurality of the said beams.

Preferably, the optical transmission bands comprise one or more of:
(i) an optical imaging band, for the optical imaging beam, using near-infrared (NIR) light, with an imaging wavelength (λ1) in a band between 700 nanometers (nm) and 3,000 nm (referred to herein as the “NIR band”), which may include a wavelength of 930, 1300, 1310 or 2000 nm;
(ii) an optical ablating band, for the optical ablating beam, in the NIR band, which may include an ablating wavelength (λ2) between 808 nm and 980 nm or 808 nm and 1100 nm, such as 1064 nm; and
(iii) an optical sensing band, for the optical sensing beam, in the NIR band, which may include a sensing wavelength (λ3) between 1300 nm and 2000 nm such as 1550 nm.

Preferably, the wavelengths of the beams are selected to differ sufficiently to remove or ameliorate inter-channel cross talk, i.e., interference of light from one of the beams into another of the beams.

The fibre optic catheter may further comprise a directional control mechanism such as a spring wire mechanism or a spiral/helical wire mechanism, preferably tensioned, to allow for the remote control of the fibre optic catheter.

Preferably, the catheter system further comprises a feedback system that controls one or more of the systems such as the optical ablating system to control burn depth during the ablation process. The burn depth may be determined from any data generated by the system, such as the optical imaging data, and can then be determined to a preselected target burn depth, a preselected damage threshold or preselected minimum tissue thickness.

Preferably, the catheter system comprises at least one optical switch adapted to switch a combined beam (for example a beam that includes the imaging beam and the ablating beam) between a plurality of different and separate optical fibres in the fibre optic catheter. Preferably, the optical switch is connected to and controlled by the electronic controller which can synchronise the switching of the optical switch with detection of the reflected imaging beam to generate images of the tissue portion. Even more preferably, the optical switch directs or routes a plurality of beams sequentially to each of the plurality of fibres for tissue imaging, ablation or other functions of the system.

The present invention also provides a method of ablating a tissue in a subject and visualising the ablation in real time using a catheter system as described herein.

More particularly, the present invention provides a method of ablating a tissue in a subject and visualising the ablation process in real time via an image of the tissue, the method comprising the steps of:
(i) positioning, adjacent to the tissue, a catheter tip assembly comprising:
   (a) an array of first optical fibres for carrying the optical imaging beam; and
   (b) an ablating means;
   wherein the catheter tip assembly is adapted to direct said beam onto the tissue portion and capture a reflected portion of the optical imaging beam from the tissue portion;
(ii) actuating the ablating means and simultaneously directing said beam onto the tissue;
(iii) actuating a first switching means to switch the optical imaging beam between a plurality of the first optical fibres and capturing the optical imaging beam reflected from the tissue;
(iv) adjusting the captured optical imaging beam reflected from the tissue to account for variations in length between the first optical fibres; and
(v) using the adjusted captured optical imaging beam from step (iv) to create the image of the tissue.

General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The term “optical beam” as used herein relates to a beam of light that carries signals and/or optical power. For example, the imaging beam can carry signals that may be used for imaging; the ablating beam can carry optical power that may be used for ablation; and the sensing beam can carry signals that may be used for sensing temperature and/or pressure at or near the leading end of the catheter tip assembly. Each beam may be directed, modulated, or transformed, and still be a beam in the sense that the same, or corresponding, signals and/or optical power are still transmitted. For example, a beam may be optically modified (e.g., optically amplified, or modulated, or shifted to a different optical wavelength), and still carry signals and power that are determined and controlled by the signals and the power before modification, and thus this may be regarded as the same beam herein.

Those skilled in the art will appreciate that the invention described herein is susceptible to variations and modifications other than those specifically described. The invention includes all such variation and modifications. The invention also includes all of the steps and features referred to or indicated in the specification, individually or collectively and any and all combinations or any two or more of the steps or features.

The present invention is not to be limited in scope by any of the specific embodiments described herein. These embodiments are intended for the purpose of exemplification only. Functionally equivalent products and methods are clearly within the scope of the invention as described herein.

The invention described herein may include one or more range of values (e.g. size etc). A range of values will be understood to include all values within the range, including the values defining the range, and values adjacent to the range which lead to the same or substantially the same outcome as the values immediately adjacent to that value which defines the boundary to the range, provided such an interpretation does not read on the prior art.

For the purposes of the present invention the terms “leading” and “following” for example in the phrases “leading end” and “following end” refer to positions relative to the position of a feature relative to the tissue being treated. “Leading” as used herein refers to a feature or part thereof that is closest or proximal to the tissue whereas “following” refers to a feature or part thereof that is furthest or distal to the tissue.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all technical terms used herein have the same meaning as commonly understood to one of ordinary skill in the art to which the invention belongs.

### DESCRIPTION OF THE PREFERRED EMBODIMENTS/EXAMPLES

The present invention will now be described more fully hereinafter with reference to the accompanying Figures, in which preferred embodiments of the invention are described. This invention may, however, be embodied in many different forms and should not be construed as limited to the embodiments set forth herein; rather, these embodiments are provided so that this disclosure will be thorough and complete, and will fully convey the scope of the invention to those skilled in the art.

Overview

Described herein are catheter systems and methods for treatment of intrabody tissues using a catheter system. The catheter systems and methods may allow for improved tissue imaging, tissue ablation, and temperature and/or pressure sensing using a single catheter in the human or animal body. The system may allow one or more of following modalities (or processes) to be provided using a single catheter: determination of vessel or heart wall proximity, thickness and character (e.g., normal pre burn, oedema post burn), determination of vessel wall contact pressure, sensing a temperature of wall tissue, burning using a focussed laser beam, and intra cardiac pacing when in the heart.

Catheter System

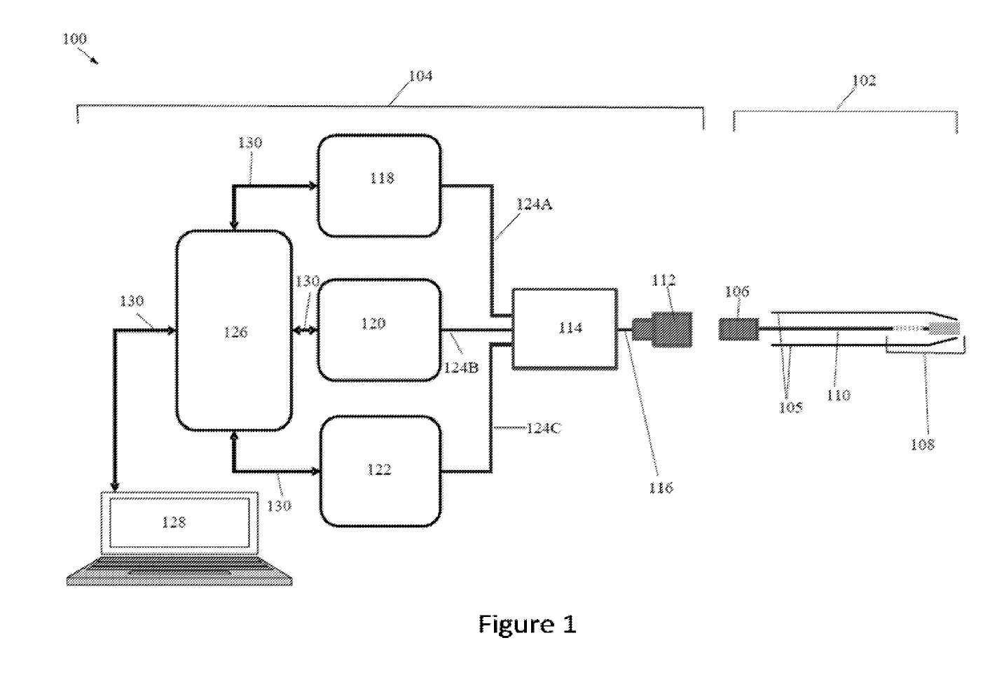

A catheter system 100, as shown in FIG. 1, includes a single-strand or multi-strand fibre optic (FO) catheter 102, configured for insertion into the body, and a catheter driver 104 that can be connected to the FO catheter 102 to carry, transmit, direct and receive an imaging beam, an ablating beam and a sensing beam, each from the catheter driver 104, into the FO catheter 102, and thus to a body portion or target area requiring diagnosis and/or treatment, e.g., by a cardiac electrophysiologist (CPE). The catheter system 100 includes a sleeve catheter 105 (or a “sheath” catheter) that mechanically supports and guides the FO catheter 102 in the body to the body portion or target area at a distal end of the sleeve catheter 105.

As shown in FIG. 1, the FO catheter 102 includes a catheter connector 106 configured to connect the FO catheter 102 to the catheter driver 104, to allow the optical imaging beam, the optical ablating beam and the optical sensing beam to be carried, transmitted and directed between the catheter driver and the FO catheter 102. The catheter connector 106 may be an optical fibre connector or adaptor. The FO catheter 102 includes a catheter tip assembly 108 at a distal end of the FO catheter 102, i.e., the end for insertion into the body, in contrast to the catheter connector 106 at a proximal end of the FO catheter 102 for connection to the catheter driver 104 outside the body. The catheter tip assembly 108 includes a pressure sensor and/or a temperature sensor in the catheter tip assembly 108. The catheter tip assembly 108 is described in more detail hereinafter.

The FO catheter 102 includes an optical conduit or fibre 110 extending between the catheter connector 106, at its proximal end, and the catheter tip assembly 108, at its distal end. The optical conduit 110 is configured to carry the imaging beam, the ablating beam and the sensing beam along the FO catheter 102 from the catheter connector 106 to the catheter tip assembly 108, and to carry the imaging beam and the sensing beam back along the FO catheter 102 from the catheter tip assembly 108 to the catheter connector 106. The optical conduit 110 may include, or may be in the form of, at least one optical fibre. The at least one optical fibre may be a single optical fibre, and the FO catheter 102 may be referred to as a single-strand FO catheter. The at least one optical fibre may include a plurality of optical fibres, or a bundle of optical fibres, and the FO catheter 102 may be referred to as a multi-strand FO catheter. In a multi-strand FO catheter: a plurality of fibres may each carry all three of the imaging beam, the ablating beam and the sensing beam; different fibres may carry different ones of the three beams; and/or one or more fibres may carry two of the three beams, while different one or more fibres may carry the other one of the three beams.

The at least one optical fibre is configured to have one or more optical transmission bands selected to carry the imaging beam, the ablating beam and the sensing beam. Exemplary selected optical transmission bands include:
(i) an imaging band, for the imaging beam, using near-infrared (NIR) light, with an imaging wavelength (λ1) in a band between 700 nanometers (nm) and 3,000 nm (referred to herein as the “NIR band”), which may include a wavelength of 930 nm or 2000 nm;
(ii) an ablating band, for the ablating beam, in the NIR band, which may include an ablating wavelength (λ2) between 808 nm and 980 nm; and
(iii) a sensing band, for the sensing beam, in the NIR band, which may include a sensing wavelength (λ3) between 1300 nm and 1550 nm.

The operational wavelengths of the imaging beam, the ablating beam and the sensing beam are selected to differ substantially to remove or ameliorate inter-channel cross talk, i.e., interference of light from one of the beams into another of the beams. For example, it is desirable to avoid any substantial leakage of light in the ablating beam entering the optical transmission wavelength(s) of the sensing beam, which would normally be of much lower power.

The catheter tip assembly 108 is in optical communication with a distal end of the optical conduit 110, and may be connected to the optical conduit 110 directly or indirectly to receive the imaging beam, the ablating beam and the sensing beam from the optical conduit 110.

A distal portion of the FO catheter 102, which includes the optical conduit 110 and the catheter tip assembly 108, has a cross-section equivalent to currently available catheter guide wires. Thus the optical conduit 110 and the catheter tip assembly 108 have cross-sectional areas equivalent to a cross-sectional area of a guide wire in the sleeve catheter 105, which is configured for use in the body by being formed of non-toxic flexible materials. In use, an operator (e.g., a CPE) may introduce the sleeve catheter 105 into the body using the Seldinger technique, which includes: first introducing the catheter guide wire into a blood vessel through a needle or trochar puncture; then threading the sleeve catheter 105 over the guide wire into the vessel and up to the point of operation or target area. As the optical conduit 110 and the catheter tip assembly 108 have cross-sections equivalent to the catheter guide wire, when the guide wire is removed (by pulling it from the body along the sleeve catheter 105), the distal portion of the FO catheter 102 can be inserted into the sleeve catheter 105 and slid to the point of operation along the sleeve catheter 105. The optical conduit 110 and the catheter tip assembly 108 may have cross-sectional diameters as small as 800 micrometers, or at least sufficient to be accommodated by a currently used catheter guide wire.

The catheter connector 106, in a proximal portion of the FO catheter 102, is exposed outside the operative field during use (by selection of a sufficient length for the optical conduit 110), and does not need to fit inside the sleeve catheter 105, and thus the catheter connector 106 may have a cross-section larger than that of a catheter guide wire. The catheter connector 106 may be 10-30 cm along the FO catheter 102 from where the FO catheter 102 enters the body, and thus at an outer end of a sterile operative area. Accordingly, components of the catheter driver 104, described hereinafter, in particular the electrical/electronic instruments, can be remote from the operating area and even within a different room. The catheter driver 104 may feed back to a live video display within direct vision of a CPE operator in the operating area.

The catheter driver 104 includes a driver connector 112 configured to connect optically to the catheter connector 106, and thus to connect optically the catheter driver 104 to the FO catheter 102. The catheter driver 104 includes an optical multiplexer 114 for combining the imaging beam, the ablating beam and the sensing beam into a single driver output conduit 116 that connects the optical multiplexer 114 and the driver connector 112, thus allowing the imaging beam, the ablating beam and the sensing beam to be directed and carried in the shared optical conduit 110. The optical multiplexer 114 can be a wavelength division multiplexer (WDM), which may be referred to as a “WDM coupler”, configured to combine the three beams into the driver output conduit 116. The driver output conduit 116 can include optical fibre equivalent to the optical fibre in the optical conduit 110, and be of the same construction as the optical conduit 110, i.e., optical fibre with the selected transmission bands.

The catheter driver 104 includes an imaging system 118 configured to generate the imaging beam for imaging tissue portions in the body, and to detect the returned imaging beam from the tissue portion to generate electronic data representing characteristics of the tissue portion for representation in a tissue image. The imaging system 118 may be an optical coherence tomography (OCT) system. The operational wavelength of the imaging system 118 is selected to correspond to the selected imaging transmission band of the optical conduit 110 for low-loss propagation of the imaging beam through the optical conduit 110. The imaging system 118 may include a currently available optical coherence tomography (OCT) imaging system that uses near-infrared (NIR) light, e.g., with a centre wavelength of 930 nm or 2000 nm. The imaging beam may be referred to as a tomography beam when it is used to generate tomographic data. The imaging beam may be referred to as a diagnostic beam when it is used to generate diagnostic data of a 2D or 3D region of the tissue portion.

The catheter driver 104 includes an ablating system 120 configured to generate the ablating beam for ablation of the tissue portion. The ablating system can be a radio frequency based system or a fibre laser system that generates an ablating laser beam at a selected wavelength for ablating tissue. When a laser based system is employed, the ablating wavelength is selected to be within the ablating band of the optical conduit 110, thus providing low-loss propagation of the ablating beam through the optical conduit 110. The ablating system 120 may include a currently available fibre laser medical ablation system with an operating wavelength of 808 nm, 980 nm and/or 2000 nm.

The catheter driver 104 includes a sensor system 122 configured to generate and to detect the sensing beam, and to determine therefrom a sensed pressure and/or a sensed temperature at and in the catheter tip assembly 108. The at least one sensing component is configured to affect (which may include modulation, or control, or changing the properties of) the sensing beam based on the sensed temperature and/or the sensed pressure at or near the catheter tip assembly. The sensing system 122 may be configured to determine the pressure and/or the temperature based on one or more wavelength shifts in the sensing wavelength of the sensing beam due to the pressure changes and the temperature changes, respectively, of the pressure sensor and the temperature sensor in the catheter tip assembly 108. The sensing wavelength shifts may be due to changes in a pressure-sensitive component and a temperature-sensitive component, each of which has an operational wavelength range. The pressure sensitive component and the temperature sensitive component may be two separate components, and each may include a plurality of elements or sub-components, which may include a plurality of pressure-sensitive sub-components for pressure-sensitive, and a plurality of temperature-sensitive sub-components for each temperature-sensitive component. The pressure sensitive component and the temperature sensitive component may each include one or more fibre gratings. The fibre gratings may include a Fibre Bragg Gratings (FBG) in the pressure sensor and a FBG in the temperature sensor. The sensing wavelength is selected to correspond to the selected sensing transmission band of the optical conduit 110, and to the operational wavelengths of the pressure sensor and the temperature sensor.

The catheter driver 104 includes a plurality of non-multiplexed conduits 124A-124C configured to carry the imaging beam, the ablating beam and the sensing beam to and from (as required) the optical multiplexer 114 and the imaging system 118, the ablating system 120 and the sensing system 122, respectively. The catheter driver 104 includes an electronic interface 126 that is in electronic communication with the imaging system 118, the ablating system 120 and the sensing system 122 to allow electronic control of the systems 118, 120 and 122, and the communication of electronic data from the systems 118, 120, 122 to an electronic computer controller 128 that is configured to control the imaging system 118, the ablating system 120 and the sensing system 122 (which may include selecting ablation parameters for the ablating system 120, including burn time and or beam intensity). The controller 128 is also configured to gather electronic data from the systems 118, 120, 122, and to display data as required (which may include displaying images using imaging data from the imaging system 118) allowing use of the catheter system 100 in diagnosis and treatment of body tissues. The computer controller 128 is connected to the electronic interface 126, and the interface 126 is connected to the systems 118, 120, 122 using electronic connections 130, which can be wired (e.g., cable) or wireless (e.g., radio frequency) data connections.

The catheter driver 104 includes a graphical video display, visible to the operator, representing: tissue proximity data, pressure data, and/or temperature data from the sensing system; and/or texture data, and/or depth data from the imaging system. As mentioned hereinbefore, components of the catheter driver 104 may be remote from the operative area, in particular the systems 118, 120, 122, the interface 126 and the controller 128; however, the controller 128 can generate data representing a combined output from all of the systems 118, 120, 122 for the live video display that the operator can observe while performing an operation. The computer controller 128 can be manipulated simply by the operator at the point of sterile catheter insertion, and switched between modalities as required, using a remote control interface of the controller 128 that provides communication between the sterile operating area and the remote controller 128.

The electronic controller 128 may include a feedback system that controls the ablating system 120 to stop the optical ablating beam if a burn depth, determined from the imaging data and/or the sensing data, is equal to or greater than a preselected damage threshold or preselected minimum tissue thickness. This may thus provide a fail-safe feedback system to prevent excessive ablation burning.

The above passages refer to the use of a sleeve catheter 105. However, it will be appreciated that a sleeve catheter is not required and the system may instead use a catheter that surrounds and supports the various components including the catheter tip assembly 108, and optical fibre 110 as well as any other components required for the system described herein. This catheter system, where sleeve catheter 105 is actually a catheter, can be inserted directly into the body and to the body portion of target area for treatment.

Catheter Tip Assembly

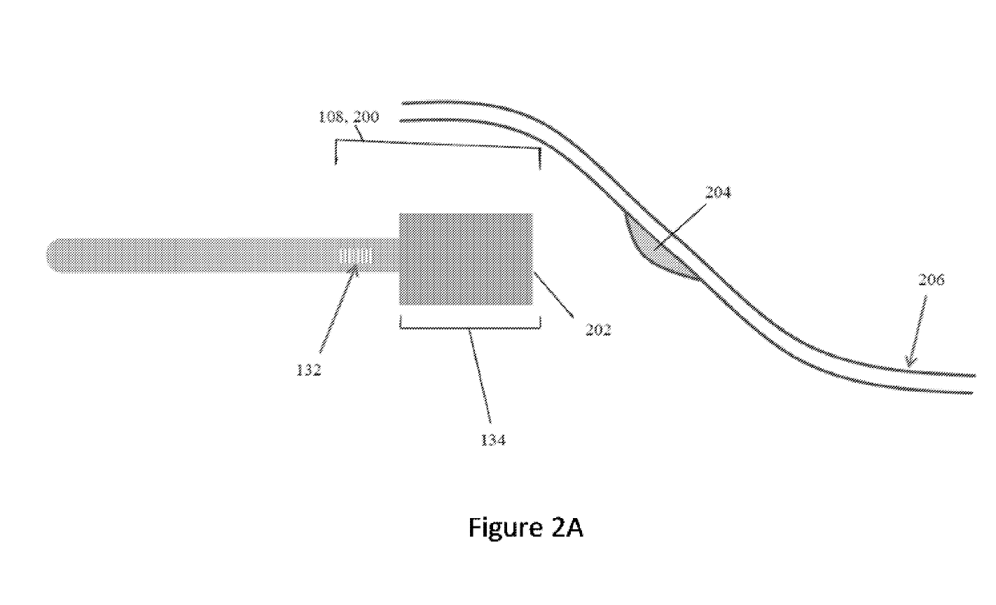

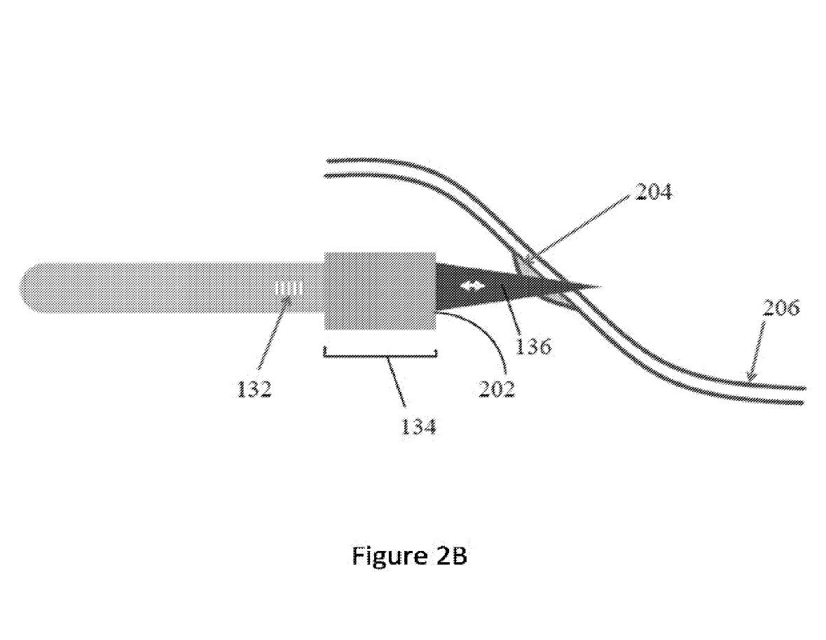

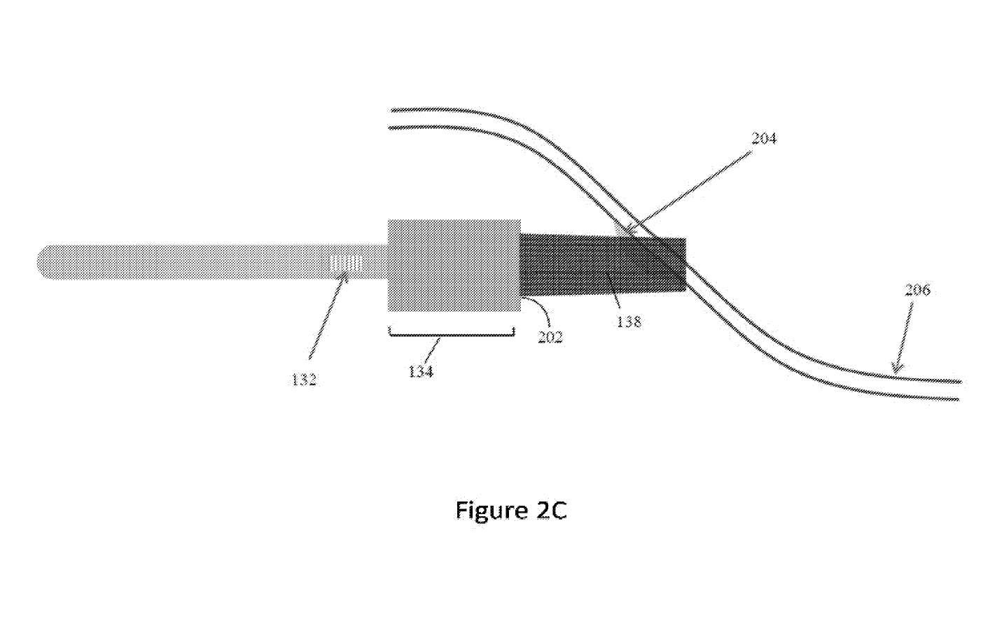

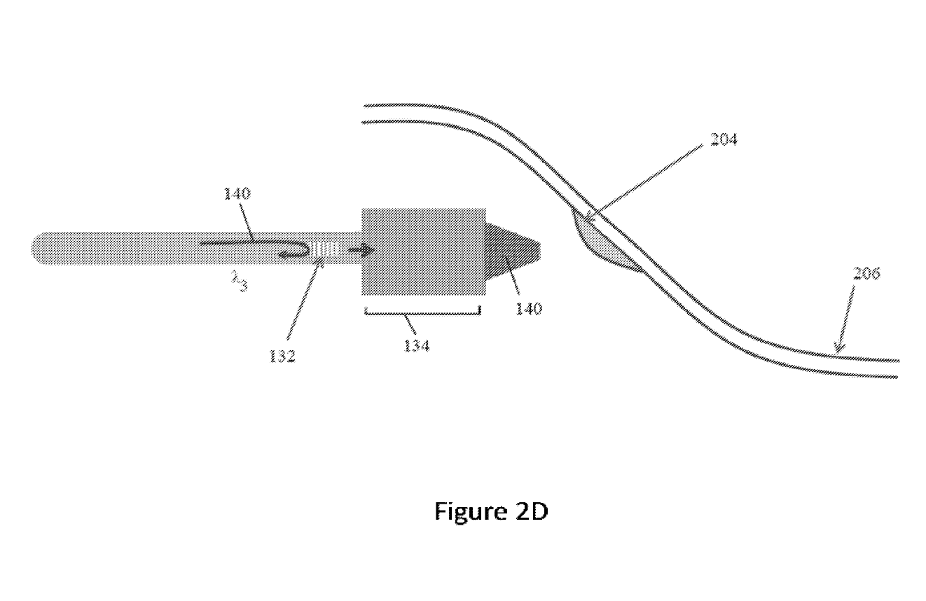

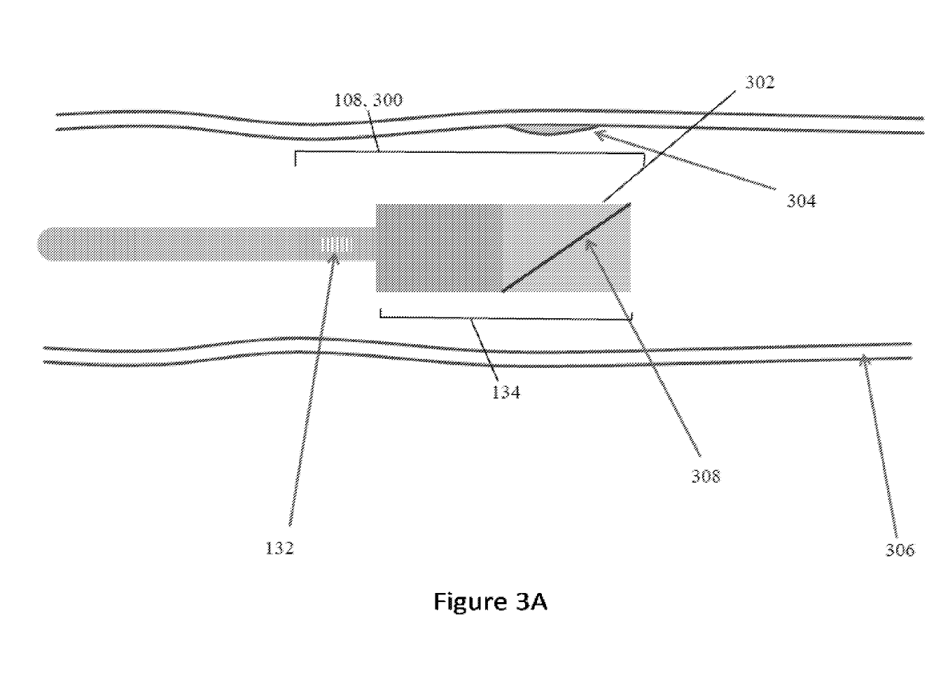

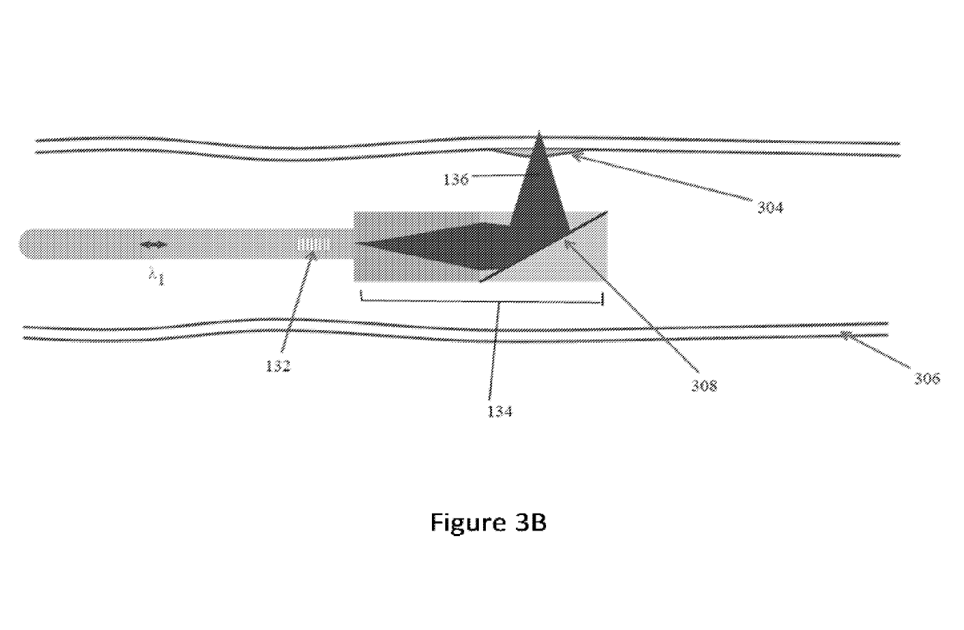

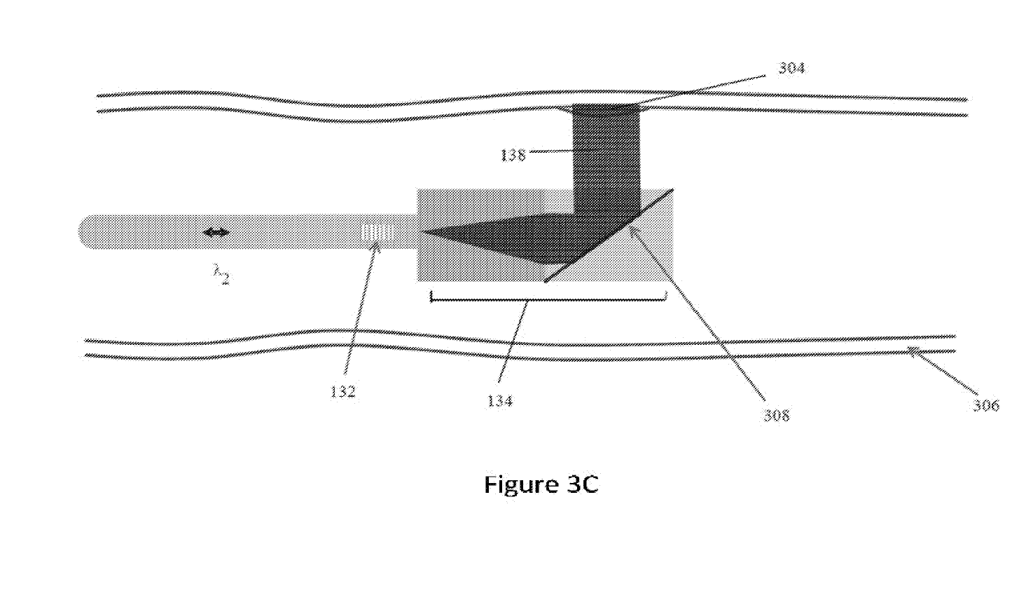

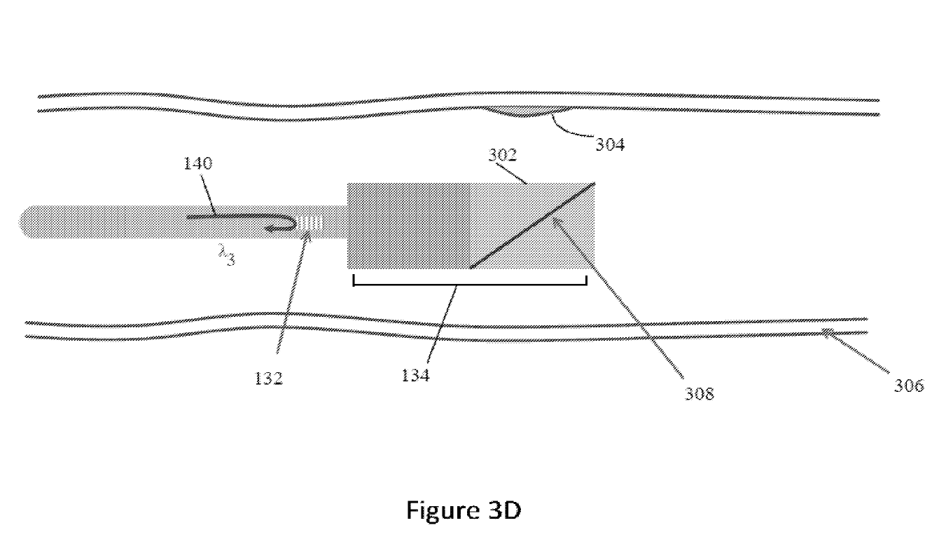

As shown in FIGS. 2A-3D, the catheter tip assembly 108 includes at least one sensing component 132 and an optical component 134 (which may be an optical directing component, and may include a plurality of optical sub-components, e.g., a compound lens and/or reflector system).

The sensing component 132 is in optical communication with the optical conduit 110, and can be embedded in the optical conduit 110. The at least one sensing component 132 may include the pressure sensor that receives the sensing beam, and modulates the sensing beam based on pressure applied, at or near the catheter tip 108, between the catheter tip assembly 108 and a selected facing tissue portion in the body. The facing tissue portion is selected by an operator of the catheter system 100, e.g., a clinician, who applies a force to the FO catheter 102 to apply pressure to the tissue portion, such as selected portion of the vessel walls. The selected tissue portion is selected based on clinical requirements, e.g., cardiac ablation to treat atrial fibrillation, or ventricular ablation to treat atherosclerosis, etc. The sensing component 132 modulates the sensing beam based on the detected pressure, thus sending an optical signal representing the detected pressure to the sensing system 122. The sensing component 132 may include the temperature sensor that detects temperature at or near the catheter tip assembly 108, e.g., due to thermal expansion and contraction of the sensing component 132, which subsequently modulates or alters the sensing beam, thus sending the sensing signal representing the temperature at the catheter tip assembly 108 to the sensing system 122. The sensing component 132 can include fibre optic pressure sensor configured for mounting at the end of an optical fibre, with cross-sectional dimensions of currently existing catheter guide wires. The sensing component 132 may include at least one fibre grating (FG) in an optical fibre of the optical conduit 110. The sensing component 132 may include a plurality of Fibre Bragg Gratings (FBGs) in materials with respective different thermal expansion coefficients, thus allowing detection of both temperature and pressure while monitoring shifts of the Bragg wavelengths of the plurality of FBGs, e.g., as described in “Progress in Electromagnetics Research Symposium”, 2005, Hanzhou, China, August 22, 26. Alternatively or additionally, the sensing component 132 can include a FBG super-imposed with a long period grating (LPG), as described in “Measurement Science and Technology”, 22, 1 (2011), 015, 202.

The optical component 134 can include at least one lens, which can be a graded index (GRIN) lens for directing both the imaging beam and the ablating beam onto the facing tissue portion for imaging (by the imaging system 118) and for treatment (by the ablating system 120). The optical component 134 can act as a focusing lens for the imaging beam and a collimating lens for the ablating beam. The amount of collimation of the ablating beam may be selected such that an insulating track of sufficient width for treatment purposes can be formed in the tissue.

In combination with the optical component 134, the imaging system 118 may produce a linear, 1-dimensional (1D) depth scan of tissue proximity, tissue character and tissue thickness at a selected spot size. The imaging spot may be scanned to generate a 2D array of the 1D depth scans, and each 1D scan may be registered (or aligned to a common reference in an X-Y plane) to construct the 2D imagine based on data representing blood flow in the heart, and/or a heartbeat.

There is no need for the optical component 134 to direct the sensing beam out of the catheter tip assembly 108 because the sensing component 132 is in the catheter tip assembly 108.

In use, the operator can use the imaging beam to observe the facing tissue portion, e.g., to determine whether treatment is required, can use the sensing beam to determine a pressure or a force applied to the facing tissue portion, can use the sensing beam to determine a temperature of the facing tissue portion, and can use the ablating beam to treat the facing tissue portion, all with little or no movement of the catheter tip assembly 108 inside the body.

Catheter Tip: End-Window Tip

As shown in FIGS. 2A-2D, the catheter tip assembly 108 can include an end window 202 configured to transmit the imaging beam and the ablating beam in a direction parallel to a longitudinal axis of the FO catheter 102 at the distal end of the FO catheter 102. The catheter tip assembly 108 with the end window 202 may be referred to as the “end-window tip 200”. In the end-window tip 200, the optical component 134 includes an axially aligned lens for focusing and collimating the imaging beam and the ablating beam, respectively, onto an end-facing tissue portion 204 that is aligned with the axial end of the catheter tip assembly 108, e.g., in or on a tissue wall 206, e.g., a vessel wall or an organ wall.

As shown in FIG. 2B, the optical component 134 can act as an imaging component for the imaging beam 136 because, at the wavelengths of the imaging beams, the optical component 134 co-operates optically with the imaging system 118 to generate images (tomographic images) of the facing tissue portion

As shown in FIG. 2C the optical component 134 directs the ablating beam 136 from the optical conduit 110 into or onto the end-facing tissue portion 204. The optical component 134 may control an amount of collimation of the ablating beam 136, and the amount of collimation may be selected based on characteristics of the ablating system 120.

As shown in FIG. 2D, the sensing beam 140 travels in the optical conduit 110 to the sensing component 132, and then back along the optical conduit 110. The sensing beam 140 does not need to extend to the end-facing tissue portion 204, or through or out of the optical component 134. As the power of the sensing beam 140 can be relatively small (e.g., a power of micro watts), any leakage of the sensing beam 140 into the tissue portion is unlikely to cause significant effects, e.g., heating.

Catheter Tip: Side-Window Tip

As shown in FIGS. 3A-3D, the catheter tip assembly 108 may include a side window 302 and a beam steerer 308 that are configured to direct the imaging beam and the ablating beam to a side facing tissue portion 304 that lies to a side of the catheter tip assembly 108, (i.e., in a radial direction, or in a direction perpendicular to a longitudinal axis of the FO catheter 102), in contrast to the axial direction of the end-facing tissue portion 204 treated by the end-window tip 200. The catheter tip assembly 108 with the side windows 302 and the beam steerer 308 may be referred to as the “side-window tip 300”.

As shown in FIGS. 3A-3D, the side-window tip 300 is similar to the end-window tip 200 in that it includes the sensing component 132 in the tip 300, and the optical component 134; however, in the side-window tip 300, the optical component 134 includes the beam steerer 308 and the side window 302, in addition to the one or more lenses. The beam steerer 308 may include a thin-film polarising beam splitter (PBS) for steering the imaging beam and the ablating beam at a right angle to the axial direction of the FO catheter 102 at its distal end. The beam steerer 308 may include a mirror, which may be a focussing mirror, e.g., a parabolic reflector.

The function and configuration of the sensing component 132 in the side-window tip 300 is the same as in the end-window tip 200 except that the sensing component 132 detects pressure applied between the side-facing tissue portion 302 and the side window 302 based on bending of flexing of the sensing component 132, and the sensing system 122 is configured to determine pressure applied between the side window 302 and the side-facing tissue portion 304 based on the magnitude and direction of the signals from the sensing component 132, and these differ from the values of the configuration parameters used in the sensor system 122 for the sensing component 132 in the end-window tip 200 because the relationship between the force applied between the end window 202 and the end-facing tissue portion 204 is different from the relationship between the force applied between the side window 302 and the side-facing tissue portion 304 and the sensor signal received from the sensing component 132. The sensing component 132 still detects temperature at and/or in the side-window tip 300.

The catheter tip assemblies described above in relation to a side window and an end window tip can be packaged into a single catheter tip assembly comprising an array of fibres such that a single catheter tip assembly can project optical beams at a variety of angles from the longitudinal axis of the catheter. Examples of this form of the invention are described later herein. It will be appreciated that by employing an array of fibres, combination of signals from multiple fibres can be selected to generate an image that the operator wants or needs in terms of visualising the ablation process.

Sleeve Catheter

The sleeve catheter 105 includes a stopper at a distal end of the sleeve catheter 105 (i.e., at the end inserted into the body to the target area) for stopping the FO catheter 102 such that the catheter tip assembly 108 is held at a selected distance from the distal end of the sleeve catheter 105. The catheter tip assembly 108 may protrude by a selected distance from the distal end of the sleeve catheter 105, depending on the type of catheter tip assembly 108. For an end-window tip 200, described hereinafter with reference to FIGS. 2A to 2D, the catheter tip assembly 108 may be stopped inside the sleeve catheter 105. For a side-window tip 300, described hereinafter with reference to FIGS. 2A to 2D, the catheter tip assembly 108 may protrude at least partially beyond the end of the sleeve catheter 105 to allow the imaging beam and the ablating beam to project outside the sleeve catheter 105. Alternatively, the sleeve catheter 105 may include a sleeve window on the end or the side of the sleeve that transmits the imaging beam and the ablating beam. The stopper may include a lock (including a cavity or projection) that receives a key (including a corresponding projection or cavity) of the catheter tip assembly 108. The catheter tip assembly 108 may be constructed to lock exactly into the stopper tip of the sleeve catheter 105, with the end window 202 just protruding beyond the sleeve catheter 105, or with the side window 302 facing a slot in the sleeve catheter 105.

The sleeve catheter 105 may be used for electronic monitoring of the heart, i.e., to detect electronic signals corresponding to a heartbeat and to act as an externally activated pacemaker if needed. The sleeve catheter 105 may include two or more electrically conductive leads along its length. For outside the body, proximal ends of the leads may be connected to an external electronic cardiac monitoring system and/or an external pace making system. For inside the body, distal ends of the leads may be connected to an internal pacemaker.

Catheter Method

The catheter system 100 performs the following catheter method using the FO catheter 102:
(i) the distal end of the FO catheter 102, including the catheter tip assembly 108, travels along inside the sleeve catheter 105 into the body until the catheter tip assembly 108 arrives at the stopper in the distal end of the sleeve catheter 105 (the sleeve catheter 105 may have been inserted into the body using a currently existing guide wire following the Seldinger Technique, and the guide wire subsequently removed);
(ii) the sensor system 122 may determine an amount of pressure and/or a temperature at or in the catheter tip assembly 108 (which may include a pressure between the catheter tip assembly 108 and the end-facing tissue portion 204, or the side-facing tissue portion 304, or a temperature of the end-facing tissue portion 204, or the side-facing tissue portion 304), and generate electronic data representing the pressure and/or temperature for the controller 128 (which may include determining an actual pressure by correcting a detected pressure using a detected simultaneous, or near-simultaneous, temperature) (“the pressure sensing step” and/or “the temperature sensing step”);
(iii) the imaging system 118 may generate and detect the imaging beam, and may generate data representing an image of the facing tissue portion (the image may be a one-dimensional, two-dimensional image, a tomographic image, and/or three-dimensional image of the tissue portion), and the image data may be processed in the controller 128 to display images of the tissue portion, which may include 1D or 2D indicators of depth profile and pressure (“the tissue imaging step”);
(iv) the external cardiac monitoring system may monitor electrical signals from the heart, which may include the heart beat, using the leads on the sleeve catheter 105;
(v) the external pace making system may send electrical signals to the heart using the leads on the sleeve catheter 105;
(vi) the ablating system 120 may generate the ablating beam to ablate the facing tissue portion based on selected values of ablation control parameters (which may include burn time and beam intensity) from the controller 128 (“the tissue ablation step”);
(vii) the imaging system 118 may generate and detect the imaging beam to generate further imaging data after the ablation to determine properties of the ablated tissue portion, including an amount of tissue that has been ablated in the ablating step using the imaging system 108, and a depth of the remaining tissue (this post-ablation data may be used to display images on the controller 128, and to generate new values for the ablation control parameters (“the further tissue imaging step”); and
(viii) the controller 128 may generate an alarm, and/or a safety cut-off signal for the ablating system 120 to shut off the ablating beam, if the controller 128 determines that the properties of the ablated tissue portion reach or correspond to a predetermined damage threshold, or a predetermined minimum tissue thickness, for the tissue.

Switched System

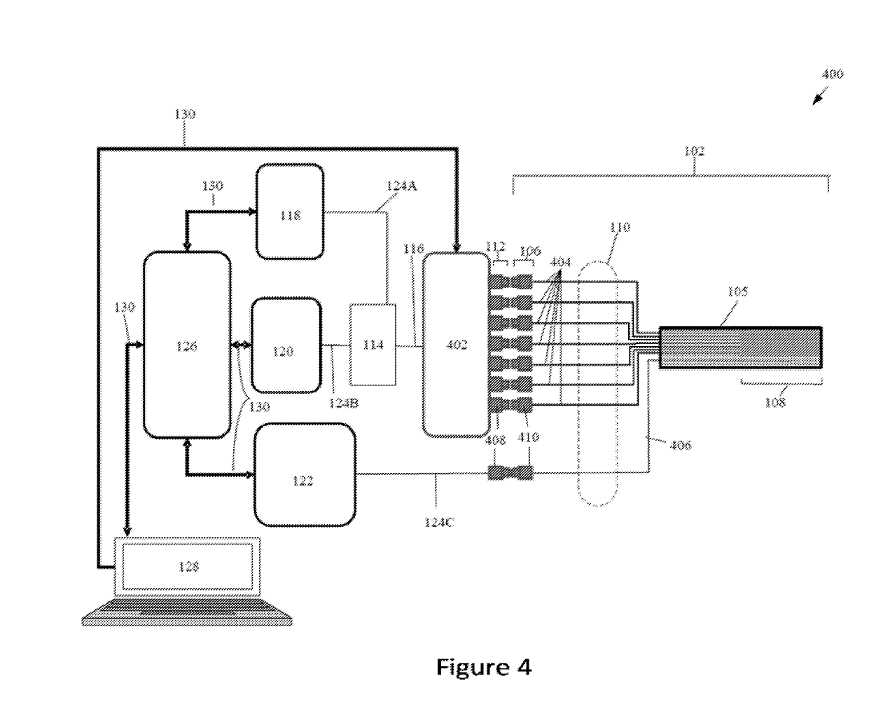

The catheter system 100 may be configured as an optically switched system 400, as shown in FIG. 4, which includes the features of the catheter system 100, and an a first switching means in the form of optical switch 402 configured to switch a combined beam (that includes the imaging beam and the ablating beam) between a plurality of different and separate optical fibres 404 in the FO catheter 102. The optical switch 402 is electronically connected to the controller 128 so that the controller 128 can synchronise the switching of the optical switch 402 with detection of the reflected imaging beam to generate images of the tissue portion. The optical switch 402 directs or routes the combined imaging and ablating beams sequentially to each of the plurality of fibres 404 for tissue imaging and ablation.

In the switched system 400, as shown in FIG. 4, the sensing beam may be directed and carried in a separate sensing fibre 406 that is separate from the combined-beam fibres 404, and parallel to them in the optical conduit 110.

In the switched system 400, the driver connector 112 includes a plurality of driver sub-connectors 408, one for each of the separate combined-beam fibres 404, and the catheter connector 106 includes a corresponding plurality of respective catheter sub-connectors 410, as shown in FIG. 4. The driver connector 112 and the catheter connector 106 also include a driver sub-connector 408 and a catheter sub-connector 410 for the sensor fibre 406. The plurality of fibres 404, 406 in the optical conduit 110 can be held in a common optical fibre casing that fits inside the sleeve catheter 105.

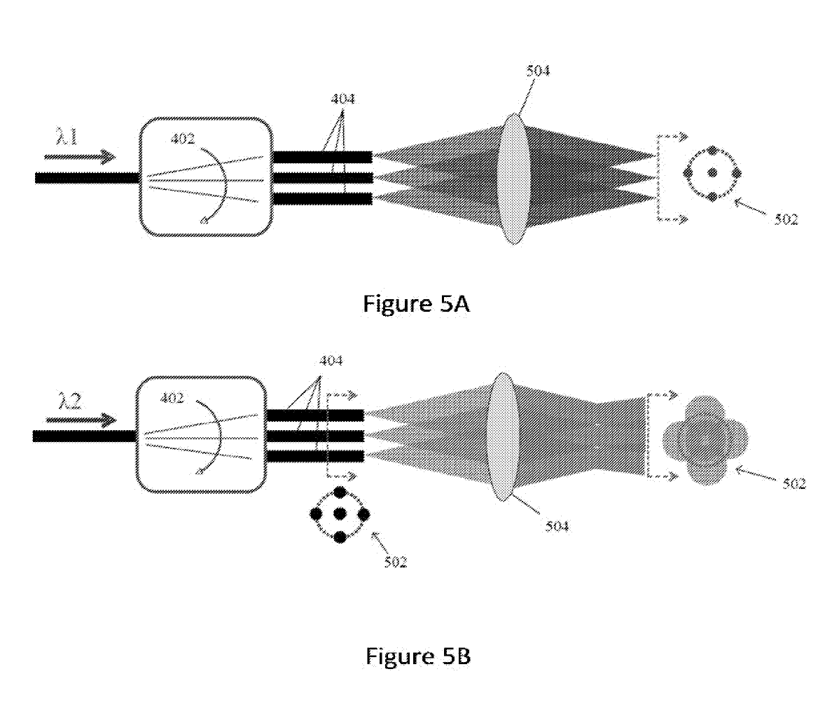

As shown in FIGS. 5A and 5B, the plurality of combined-beam fibres are arranged in an array or a pattern at their distal ends so that light is delivered to the optical component 134 according to this pattern. The pattern may include a five-point pattern 502, as shown in FIGS. 5A and 5B, with four of the combined-beam fibres 404 arranged with end-points on a circle around a central end point of one of the combined-beam fibres 404. The optical pattern may include two or more points around the circle and a central point, and may include more than five points. The optical switch 402 is controlled by the controller 128 to switch the combined beam (including the imaging beam and the ablating beam) sequentially between the fibres 404 arranged in the pattern, thus applying the combined beam according to the pattern.

In the switched system 400, the optical component 134 may include a lens or a lens relay configured to focus the imaging beam to a smaller spot on the tissue portion than the optical ablating beam (i.e., such that, for each of the combined-beam fibres 404, the spot of the optical imaging beam on the tissue portion is smaller than the spot of the optical ablating beam on the tissue portion), as shown in FIGS. 5A and 5B. The lens or lens relay 504 may be configured to focus the imaging beam to a spot size (or beam diameter) of about 10-20 microns, and to expand the ablating beam spot size (or beam diameter) to about 200-500 microns). The lens or lens relay 504 is configured to have different focal lengths for the different wavelengths of light in the different beams, thus focusing the beam more tightly than the ablating beam.

The switched system 400 allows for three-dimensional (3D) scanning using an OCT system as the imaging system 118. The imaging spot pattern can cover an imaging area of several millimetres (mm) in diameter. The light wavelength used for the imaging beam is generally quite different from the wavelength used for the ablating beam (which may be infrared). The difference in refraction with wavelength may allow the focus of the fixed lens in the catheter tip assembly 108 to blur the ablating beam into individual overlapping spots forming a larger ablating area than that of the more sharply focussed imaging spots. Thus the ablating beam may cover a whole area for treatment, while the imaging beam may image the selected points within the treatment area. The detected imaging points can be digitally combined to create an image of the treatment area: this image may be a coarsely pixelated image, or may be digitally smoothed for presentation to the operator. The 3D imaging data may be generated repeatedly, or continuously, interspersed with short periods of ablating activity (milliseconds of time). Thus the degree of burning can be repeatedly or continuously assessed by the operator using the imaging system 118 during an ablating procedure. The FO catheter 102 may be a passive, optically activated, interchangeable apparatus, and thus may be capable of low-cost, mass production.

Rotary Tip

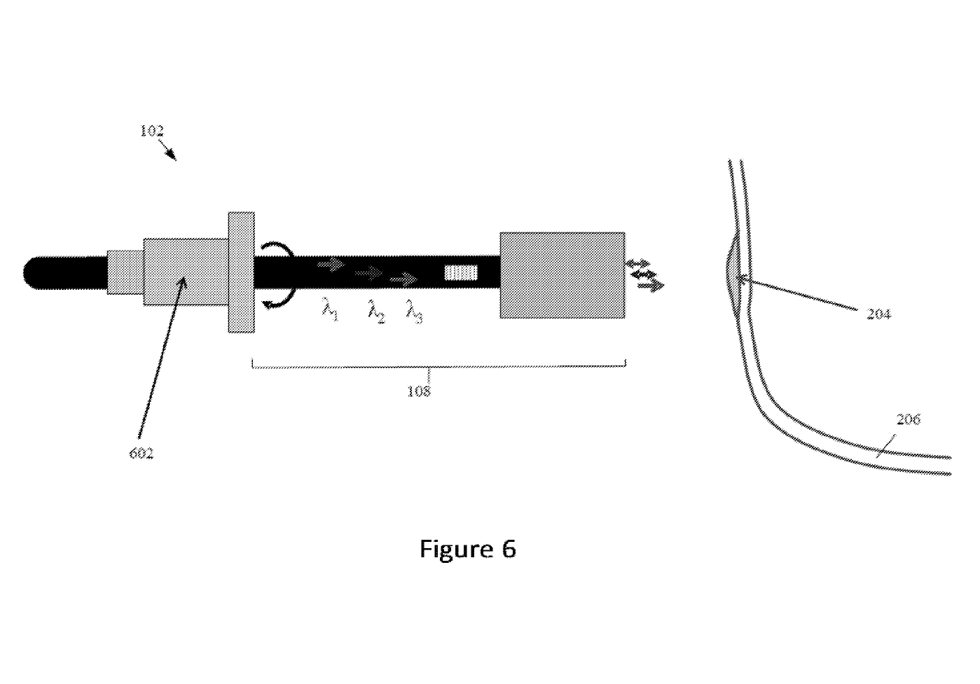

The optical catheter 102 may include a rotary tip provided by a rotary joint 602 between the optical conduit 110 and the catheter tip assembly 108, as shown in FIG. 6. The rotary joint 602 may be an existing fibre optic rotary joint that rotates the catheter fibre, and thus the catheter tip assembly 108, to provide rotary scanning for the imaging system 118.

The catheter system 100 may include a stepper motor for pushing the FO catheter 102 through the body (which may be in a vessel, or the sleeve catheter 105) under control of the controller 128, while the rotary joint 602 is controlled such that signals and data from the rotating and/or longitudinally moving catheter tip assembly 108 are reconstructed by the imaging system 118 to form useful image data.

It will be appreciated that other parts of the catheter system can be adapted to be rotated. For example, the catheter tip assembly may be adapted for rotation or the platform that can be located inside the catheter tip assembly can be adapted for rotation. Rotation of these parts of the system can perform a rotary scan and/or allow fixed fibres in an array to be located adjacent a lens or another optical directing component that is moved during the rotation of the system part. For example, the platform inside the catheter tip assembly could comprise a plurality of different lens fixed therein that upon rotation of the platform (or catheter tip assembly) are positioned adjacent to and moved between a plurality of fixed fibres in the array.

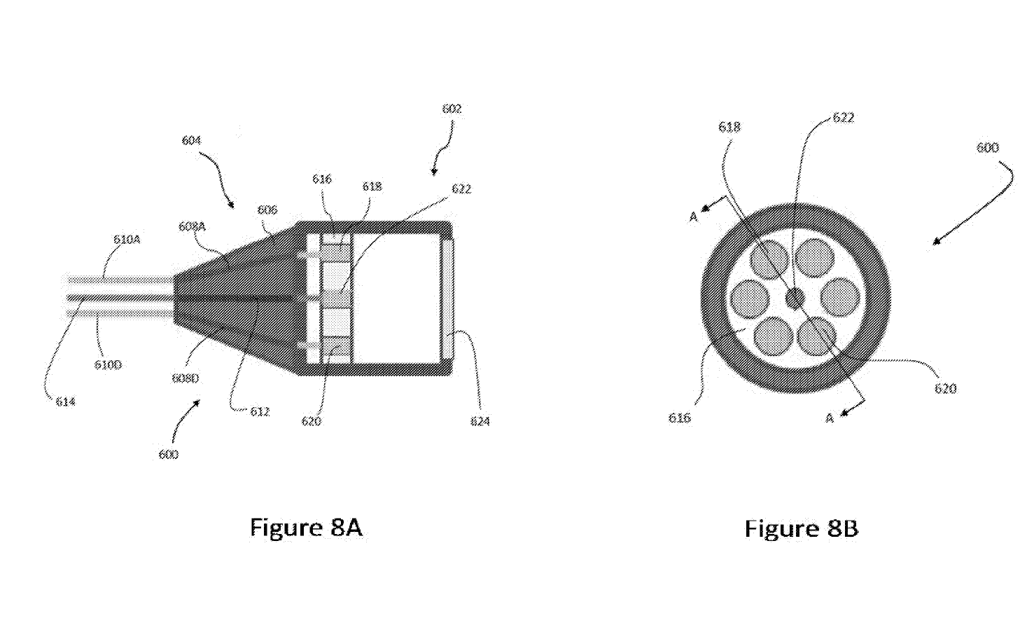

A catheter tip assembly for a catheter system according to one embodiment of the present invention, generally indicated by the numeral 600 is depicted in FIGS. 8A and 8B. The catheter tip assembly 600 comprises a generally circular cross sectional shape and defines a leading end 602 and a trailing end 604. Trailing end 604 includes a pigtail 606 for retaining the leading ends 608A, 608D (only two are shown in FIG. 8A) of a plurality of first conduits for optical imaging beams in the form of an array of six optical fibres 610A, 610D (only two are shown in FIG. 8A). The pigtail 606 also retains the leading ends 612 of a second conduit for an optical ablating beam in the form of optical fibre 614. Optical fibre 614 is multi-directional to allow for reflected light to be captured for further processing.

Each leading end including leading ends 608A, 608D (only two are shown in FIG. 8A) terminate at a perforated platform member 616 including a plurality of lenses, six in total (only 618 and 620 are shown) for focusing or narrowing an OCT beam passed therethrough.

The leading end 612 of optical fibre 614 also terminates at platform member 616 that also includes lens 622 for spreading or diverging an ablating beam passed therethrough.

The leading end 602 of the catheter tip assembly 600 also includes a glass aperture 624 positioned nearest the tissue to be ablated, when in use, and through which the beams pass before reaching the tissue.

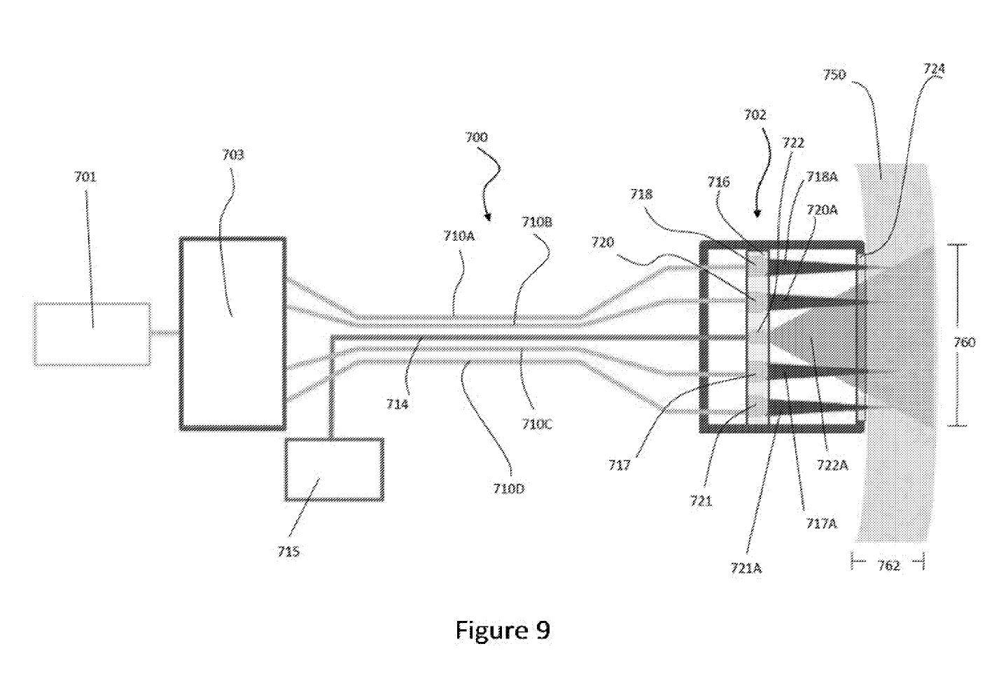

FIG. 9 depicts another embodiment of a catheter system of the present invention. The catheter system, generally indicated by the numeral 700, is shown in use and located adjacent to a tissue 750 requiring treatment.

The catheter 700 includes a plurality or array of first conduits or fibres (four in total) 710A-710D for optical imaging beams with a wavelength of 1310 nm generated by an optical coherence tomography system 701 that includes an optical switch 703, and second conduit 714 for an optical ablating beam. Optical fibre 714 is multi-directional to allow for reflected light to be captured for further processing. In use, the first and second conduits or fibres and the catheter tip assembly (see below) would be retained inside a sleeve catheter (not shown).

The fibre optic catheter 700 includes a catheter tip assembly 702 of a similar form and configuration to that shown in FIGS. 8A and 8B and includes a perforated platform member 716 including a plurality of lenses, four in total (717, 718, 720, 721) for focusing or narrowing the OCT beam passed therethrough to form optical imaging beams 717A, 718A, 720A and 721A that are directed onto tissue 750 at or near the site of ablation (see below).

The leading end of optical fibre 714 also terminates at platform member 716 that includes lens 722 for spreading or diverging an ablating beam with a wavelength of 1064 nm generated by laser 715. The ablating beam 722A is directed onto the target tissue to perform a controlled ablation of the tissue at the site of ablation to a depth 762 of about 2-2.5 mm with a width 760 of about 2.5 mm.

The catheter tip assembly 702 also includes a glass aperture 724 positioned nearest the tissue to be ablated, when in use, and through which the beams pass before reaching the tissue.

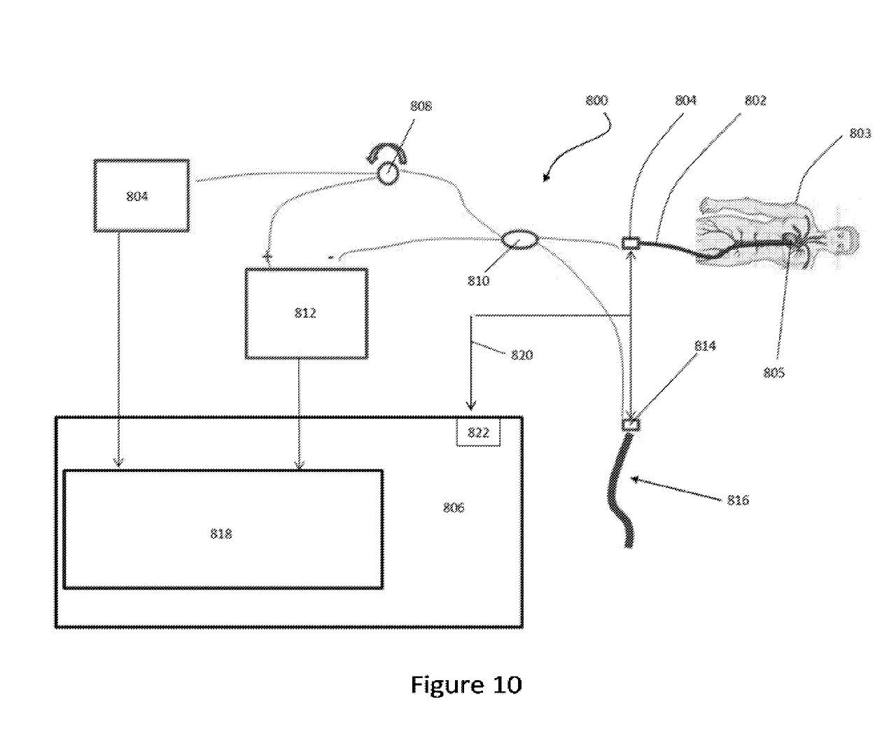

FIG. 10 is a schematic representation of another catheter system according to the present invention. The system generally indicated by the numeral 800 comprises a catheter 802, similar to that depicted in FIG. 9, inserted into the heart 805 of a patient 803 via the femoral artery to a predetermined treatment site in the heart 805 that is in need of tissue ablation. The catheter includes a catheter tip assembly according (not shown).

A tuneable light source 804 including a driver under the control of an electronic controller in the form of computer 806 configured to apply swept source OCT delivers an optical imaging beam to the treatment site via a multi-port circulator 808 and a 50/50 coupler 810. The 50/50 coupler 810 splits the optical imaging beam into first and second identical optical imaging beams. One of these beams continues via first optical switch 804 and the catheter 802 and catheter tip assembly (not shown) to the treatment site in the heart 805 and the other beam is transmitted via a second optical switch 814 to a reference means 816 comprising an arrangement of optical fibres of known length that terminate in an arrangement of microfibre mirrors or lenses (not shown).

Light reflected from the optical imaging beam at the treatment site in the heart 805 is captured by the catheter tip assembly and light reflected by the reference means 816 are transmitted to a photodetector 812 and in turn transferred to data acquisition means 818 that forms part of computer 806. Image data 820 from the reference means 816 is received by transceiver 822 and used to adjust image data received at the data acquisition means 812 from the treatment site to account for variations in optical fibre lengths between different catheters. This ensures that the image data from each optical fibre in the catheter 802 is seen as a standardised signal in appropriate phase with the fibres of varying lengths.

An optical ablation beam (not shown) through a single central fibre or through multiple dedicated fibres is also generated and delivered to the same treatment site. This beam is also under control of the computer 806. It will be appreciated that a radio frequency ablation means could be used instead of in addition to the optical ablation beam.

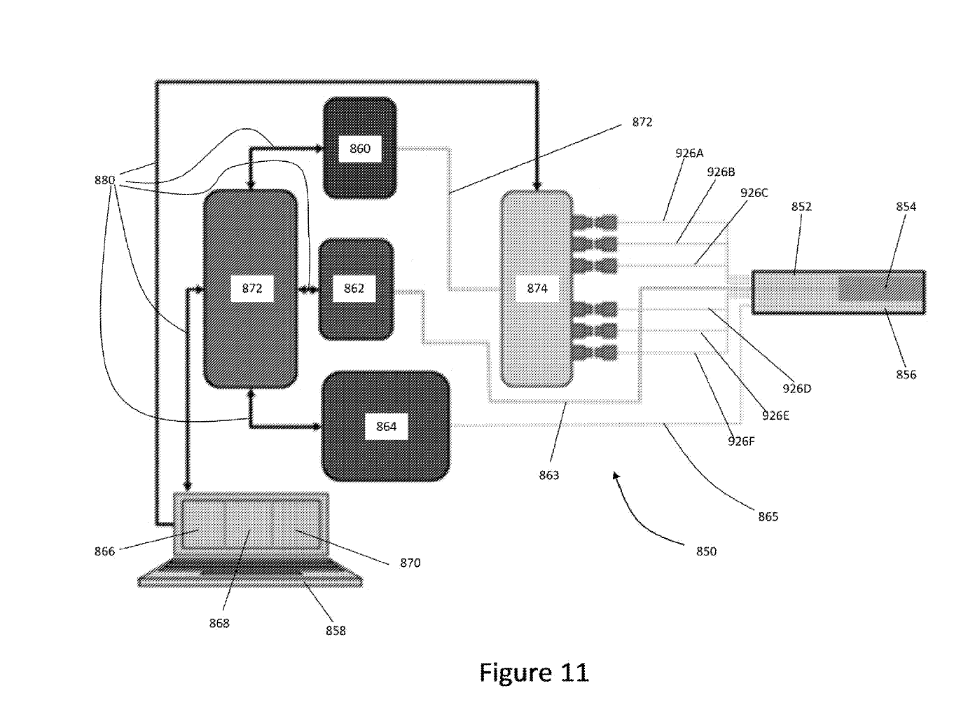

FIG. 11 is a schematic layout of a catheter system according an embodiment of the present invention. The system generally indicated by the numeral 850 comprises a catheter in the form of a disposable fibre optic catheter tip assembly 852 that includes a lens relay 854 for an array of optical fibres and an in-fibre microstructure for pressure and temperature sensing 856. These features could correspond to those depicted in other figures herein or as otherwise described herein.

The catheter system 850 is controlled by an operator via a controller in the form of a computer 858 that controls the three main functions of the system—the OCT system 860, laser 862 and an optional pressure/temperature sensor system 864. It is preferred that the computer 858 includes a GUI with components covering each of these main functions 866, 868 and 870 for the OCT 860, laser 862 and pressure/temperature system 864, respectively and operates via a printed circuit board interface 872.

The OCT system 860 generates an optical beam at a predetermined wavelength that is transmitted along optical fibre 872 to optical switch 874 that splits the optical beam into six channels that carry the optical beam to the lens relay 854 and, in turn, out from the leading end of the catheter tip assembly 852. Reflected optical beams from the treatment site are captured by the catheter tip assembly 852 and fed back to the OCT system 860 for processing to form an image of the site adjacent to the leading end of the catheter tip assembly 852 that can be presented to an operator via computer 858.

Similarly, laser 862 generates an optical ablation beam at a predetermined wavelength that is transmitted along optical fibre 863 to the lens relay 854 and in turn out from the leading end of the catheter tip assembly 852 to the treatment site to ablate a treatment site adjacent thereto. The optical pressure/temperature sensor system 864 also generates an optical beam at a predetermined wavelength that is transmitted along optical fibre 865 to the lens relay 854. One or more of the optical fibres may incorporate a lens at the lens relay 854 to alter the angle of the beam as it leaves the catheter tip assembly 852.

Various components in the catheter system may be electrically interconnected by electrical conduits 880.

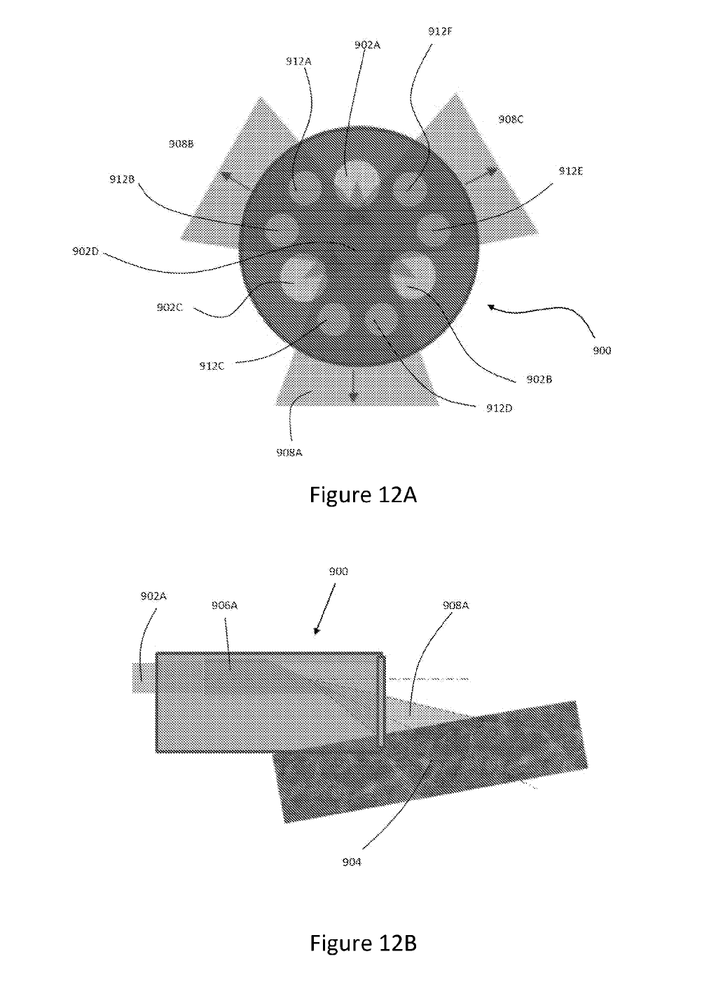

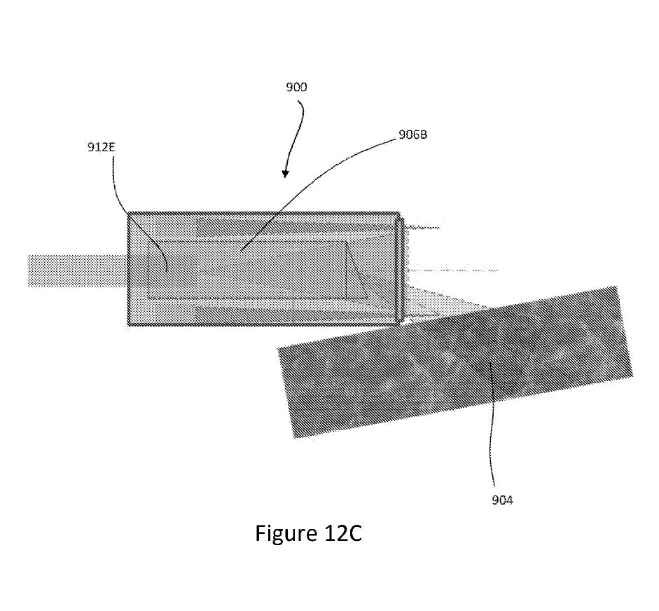

FIGS. 12A-12C illustrate one example of a catheter tip assembly that forms part of a catheter system according to one embodiment of the present invention. The catheter tip assembly, generally indicated by the numeral 900 can be used in the system illustrated in FIGS. 9-11 and includes an ablating means in the form of a laser that emanates from a fibre laser (not shown) and is delivered to the catheter tip assembly 900 by optical fibres in the form of GRIN fibres 902A-902D. The array of four fibres 902A-902D can be controlled independently to customise the delivery of the laser ablation to the site of treatment 904.

The ends of each of the fibres 902A-902C incorporate a prism 906A (only one is shown in FIG. 12B) that acts to divert the laser onto the tissue at a predetermined angle whereas fibre 902D emits the laser directly from its end with no diversion. The diverted laser beams from each of fibres 902A-902C are shown in FIG. 12A as 908A-908C are FIG. 12B shows the independent operation of fibre 902A that emanates ablation beam 908A to the site of treatment 904.

The catheter tip assembly 900 also includes an array of first optical fibres in the form of six fibres 912A-912F for carrying an optical imaging beam generated by an OCT system (not shown). FIG. 12C shows the beam from fibre 912E that passes through a prism 906B integrally provided at the leading end of the fibre 912E to divert the beam onto the site of treatment 904. A reflected portion of the beam from fibre 912E is captured by the catheter tip assembly 900 and returned to the OCT system for processing and to enable the ablation process to be viewed by an operator.

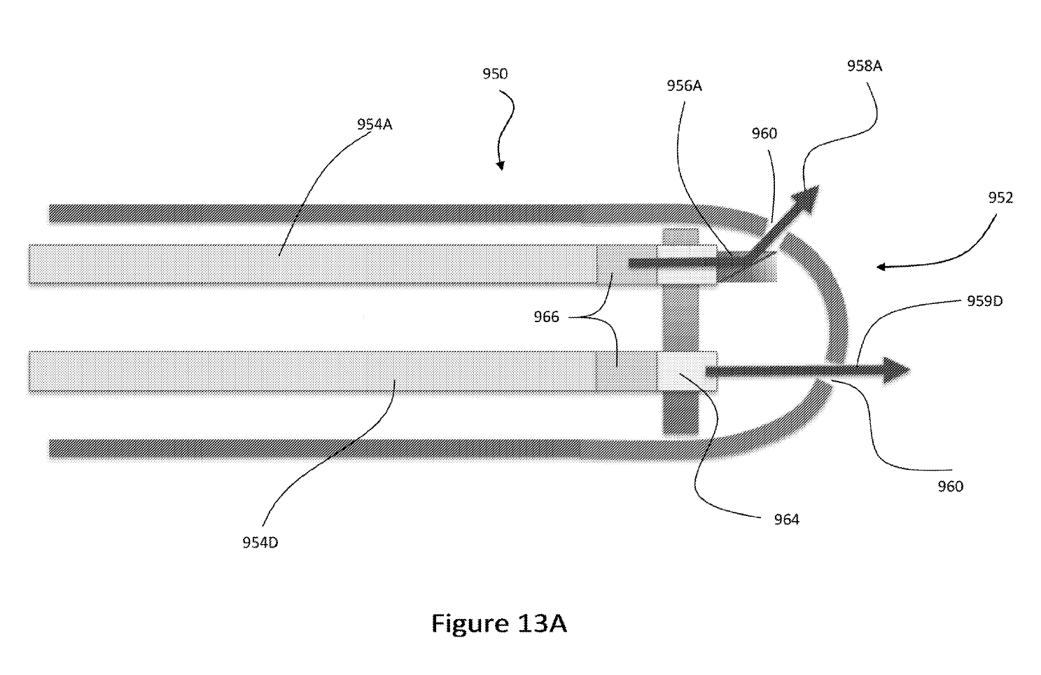

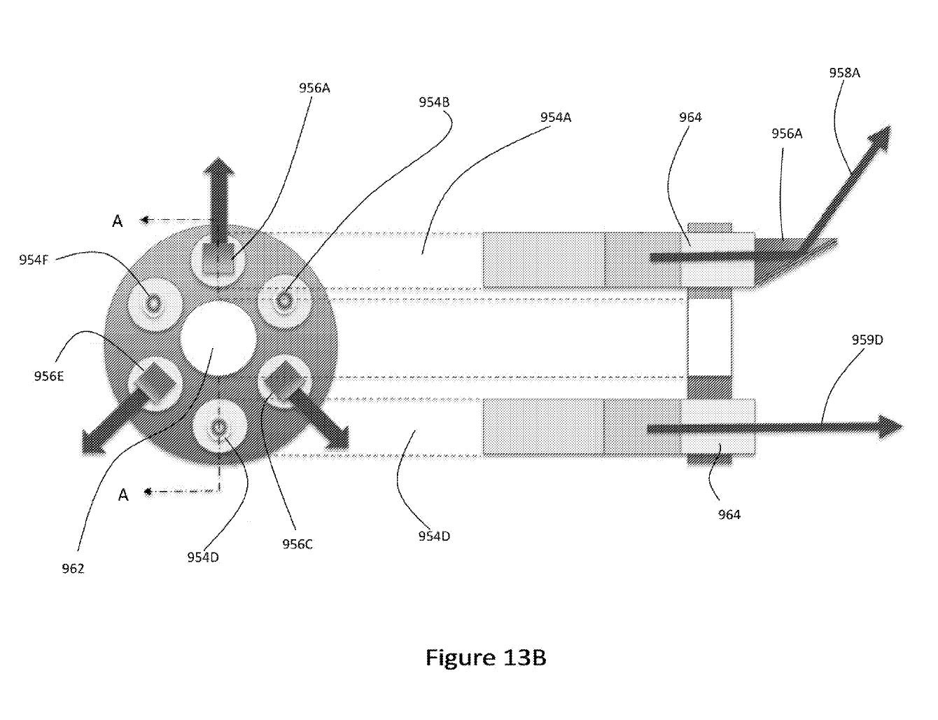

FIGS. 13A and 13B illustrate an example of a catheter tip assembly that forms part of a catheter system according to one embodiment of the present invention. This example shows the ability for the catheter system to address tissue in the axis of the catheter tip assembly and tissue in contact with the side of the catheter tip assembly. The catheter tip assembly, generally indicated by the numeral 950 can be used in the system illustrated in FIGS. 9-11 and includes an ablating means in the form of an RF ablation electrode (not shown) that heats the outer surface of the catheter tip assembly at its leading end 952. Although not shown the RF ablation electrode or power source cable may be supported in the central lumen 962 in the body of the catheter tip assembly 950. Central lumen 962 can also support other components such as guide wires, pacing, ECG leads and saline injection conduit.

The catheter tip assembly includes an array of first optical fibres for carrying an optical imaging beam in the form of six optical fibres 954A-954F. Three of these fibres (954A, 954C and 954E) include prisms 956A, 956C and 956E that allow for the optical beam to emitted at an angle of 45°—only one beam 958A is shown. These prisms may use a thin film coating for beam reflection. The other three fibres (954B, 954D and 954F) emit the beam straight ahead—only one beam 959D is shown. All of fibres 954A-954F include a GRIN fibre portion 964 and a hollow fibre portion 966. The catheter tip assembly 950 also includes IR transparent windows 960 to allow the beams through the leading end 952 of the catheter tip assembly 950.

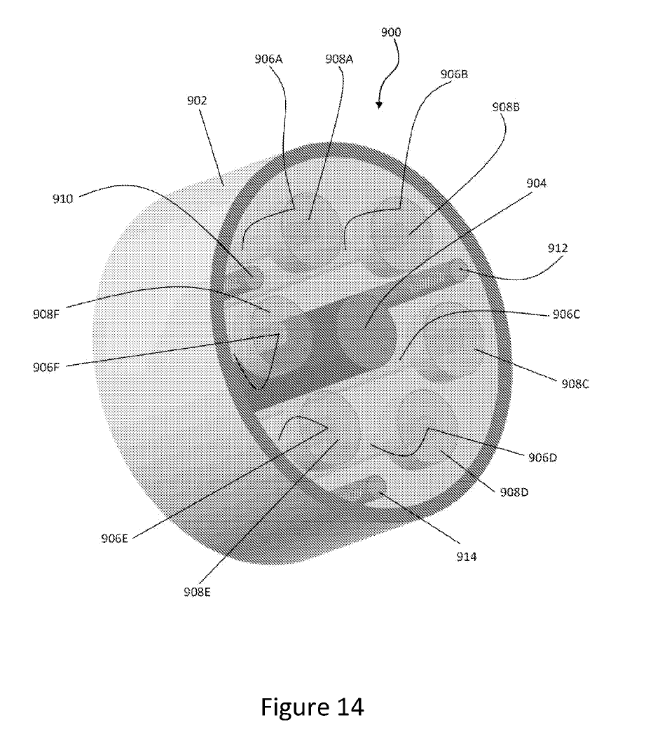

FIG. 14 illustrates a part of another example of a catheter tip assembly that forms part of a catheter system according to one embodiment of the present invention. The portion of the catheter tip assembly, generally indicated by the numeral 900, shows the arrangement of various components within a flexible catheter body 902 and can for part of a catheter tip assembly used in the system illustrated in FIGS. 9-11. The portion 900 includes an ablating means in the form of an RF ablation electrode 904 that is centrally located and acts to heat the outer surface of the catheter tip assembly at its leading end. Around the RF ablation electrode is arranged an array of first optical fibres for carrying an optical imaging beam in the form of six optical fibres 906A-906F, all of which include prisms 908A-908F that allow for the optical beam to emitted at an angle. These prisms may use a thin film coating for beam reflection. The catheter tip assembly 900 also includes three additional optical fibres 910, 912 and 914 that can function as temperature and or pressure/force sensors.

### APPLICATIONS

Embodiments of the present invention may provide effective results when used for procedures, e.g., cardiac ablation. In cardiac ablation, embodiments of the present invention may combine the functions of burning, pace making, monitoring, and tissue imaging into a single catheter, thus reducing the number of catheter insertions. Embodiments may allow for more accurate and quicker ablation performance, and may reduce requirements for repeat ablations on the same patient. Embodiments may reduce the total cost of catheters required for an example procedure.

Using an optical beam as the ablating beam may be more accurate and less damaging than using radio frequency (RF) ablation provided by currently existing medical ablation systems, due to more accurate control of width, depth, position and intensity of the burn.

Compared to existing techniques, having the FO catheter 102 fit inside the sleeve catheter 105 configured for existing guide wires may simplify the procedure of operations such as ablation of heart tissue to treat atrial fibrillation.

A field of view provided by the imaging system 118 and the optical component 134 may be up to or larger than 1 cm², thus providing more accurate area and depth information via the controller 128 for the operator prior to and during the tissue ablation step compared to currently existing systems. The catheter system 100 may enable more accurate ablation of difficult tissue sites, e.g., the ridge between the left superior pulmonary vein and the left atrial appendage, and the imaging of ablated tissues may allow for minimisation of post-operative arrhythmia, e.g., due to damage of surrounding tissues.

The integrated pressure sensor may ensure that the tip of the catheter is in adequate contact with the heart or vessel wall at the time of ablation/burning, with the electronic real-time fail-safe assisting to provide optimal accuracy.

The integrated temperature sensor may allow for determination and monitoring of the tissue temperature before, and during, and after the ablation/burning, e.g., to avoid or ameliorate undesirable injuries.

The FO catheter 102 may be formed to be inexpensive, reusable, and/or recyclable to allow the FO catheter 102 to be essentially one-use and disposable, while allowing re-use of the catheter driver 104. With respect to disposable catheters it will be noted that the inclusion of the means for processing the reflected portion of the optical imaging beam to account for variations in length between the first optical fibres that forms part of the catheter system allows for new disposable catheters attached to catheter system, employing an array of fibres, to be conveniently and efficiently calibrated to account for differences in optical fibre lengths in the array.

The data from the imaging system 118 can be used to determine ablation intensity and ablation duration e.g., based on the observed tissue depth of the facing tissue portion.

The FO catheter 102 need not include any electronic components that may be problematic in the body in certain applications (e.g., due to undesirable interactions between electrically conductive components and the body), and thus may be referred to as an “all-optical FO catheter”. The FO catheter 102 may be used in place of the standard catheter in the technique of Intracardiac Ablation. The FO catheter 102 may be usable, with minimal modification, to access, visualize and deliver controlled ablation to many other accessible organs and tissues within the body. However, it will be appreciated that if RF ablation is applied the catheter must further comprise an electrical conductor, such as a wire, from the RF generator to the application point on the catheter tip assembly that may be formed as a gold band or cap.

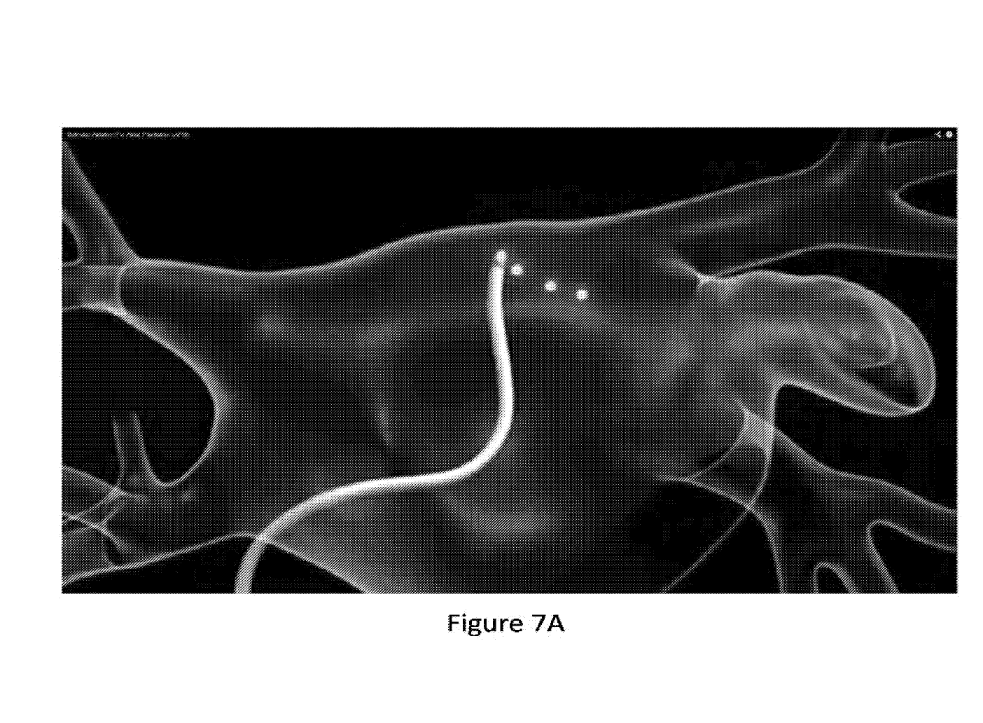

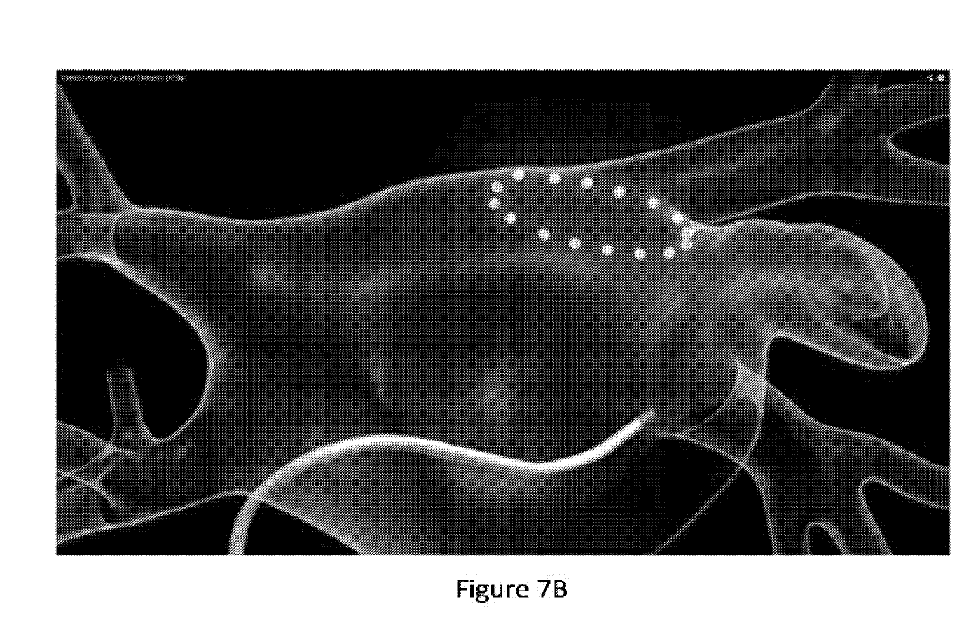

The catheter system 100 may be used for controlled tissue ablation inside an organ, including ablating tissue at local burn spots in a circle on a surface of the organ, as shown in FIGS. 7A and 7B.

The foregoing is illustrative of the present invention and is not to be construed as limiting thereof. Although a number of exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims.

1-57. (canceled)

58. A catheter system for ablating a tissue portion of a body and visualising the ablation in real time, the system comprising:
(i) an optical coherence tomography (OCT) system for generating an optical imaging beam;
(ii) a catheter including a catheter tip assembly comprising:
(a) an array of first optical fibres for carrying the optical imaging beam; and
(b) an ablating means;
wherein the catheter tip assembly is adapted to direct said beam onto the tissue portion and capture a reflected portion of the optical imaging beam from the tissue portion;
(iii) a first switching means for switching the optical imaging beam between a plurality of the first optical fibres in the array; and
(iv) a means for processing the reflected portion of the optical imaging beam to account for variations in length between the first optical fibres.

59. A catheter system according claim 58 wherein the means for processing the reflected portion of the optical imaging beam to account for variations in length between the first optical fibres comprises a reference data source.

60. A catheter system according to claim 59 wherein the reference data source comprises a second array of first optical fibres for carrying the optical imaging beam.

61. A catheter system according to claim 60 wherein said means further comprises a second switching means for switching the optical imaging beam between a plurality of the first optical fibres in the second array.

62. A catheter system according to claim 59 wherein the means for processing the reflected portion of the optical imaging beam to account for variations in length between the first optical fibres comprises software including an algorithm that calibrates the reflected portion of the optical imaging beam based on the reference data source.

63. A catheter system according to claim 61 comprising a splitter arranged to split the optical imaging beam into first beam directed to the first switching means and second beam directed to the second optical switching means.

64. A catheter system according to claim 58 wherein the array of first optical fibres comprises at least 2 optical fibres.

65. A catheter system according to claim 64 wherein the array of first optical fibres comprises between 2 and 20 optical fibres.

66. A catheter system according to claim 65 wherein the array of first optical fibres comprises between 2 and 10 optical fibres.

67. A catheter system according to claim 66 wherein the array of first optical fibres comprises between 2 and 6 optical fibres.

68. A catheter system according to claim 58 wherein at least one of the first optical fibres further comprises an optical directing component and wherein the optical directing component is adapted to deflect a beam emanating from the at least one of the first optical fibre by (a) about 30°-60°; or (b) about 45°; or (c) less than or equal to 90°; and the optical directing component is a lens

69. A catheter system according to claim 58 wherein the ablating means is an optical ablating beam carried by either one or more of the fibres in the array of first fibres together with the optical imaging beam.

70. A catheter system according to claim 58 wherein the first switching means is adapted to switch a combined beam which includes the optical imaging beam and the optical ablating beam sequentially between a plurality of the first optical fibres.

71. A catheter system according to claim 58 wherein the ablating means is an optical ablating beam carried one or more second fibres separate to the first fibres, the one or more second fibres extending through the catheter tip assembly.

72. A catheter system according to claim 58 wherein the ablating means comprises (a) a member heated by electricity or radio frequency waves. The catheter tip assembly and adapted to transmit heat energy form a source to the tissue portion; or (b) a radio frequency ablation electrode that extends through the catheter tip assembly.

73. A catheter system according claim 58 wherein the catheter tip assembly further comprises one or more of (a) a sensing component; (b) electrical leads for connection to a cardiac pace maker; (c) a fluid channel of flushing system; (d) a directional control mechanism to enable remote movement control of the fibre optic catheter; (e) a means for emitting ultrasound waves;
and (e) at least one magnet.

74. A method of ablating a tissue in a subject and visualising the ablation process in real time via an image of the tissue, the method comprising the steps of:
(i) generating an optical imaging beam using an optical coherence tomography (OCT) system
(ii) switching the optical imaging beam through a first array of a plurality of first optical fibres carried in a catheter tip assembly to illuminate a portion of tissue and capturing a reflected imaging beam being reflected from the portion of tissue beam;
(iii) switching the optical imaging beam through a second array of the first optical fibres and using data generated from the second array to facilitate adjustment of the reflect image beam to account for variations in length of fibres in the first array;
(iv) using the adjusted reflected image beam from step (iv) and the OCT system to create the image of the tissue;
(v) directing an ablation beam through the catheter tip assembly to ablate a portion of tissue imaged by the OCT system.
